(19) United States
(12) Patent Application Publication
(10) Pub. No.: US 2021/0023165 A1
(43) Pub. Date: Jan. 28, 2021

(54) OPHTHALMIC DRUG SUSTAINED RELEASE FORMULATION AND USES FOR DRY EYE SYNDROME TREATMENT

(71) Applicant: Mati Therapeutics, Inc., Austin, TX (US)

(72) Inventors: Deepank Utkhede, Surrey (CA); David J. Wiseman, Surrey (CA)

(73) Assignee: Mati Therapeutics, Inc., Austin, TX (US)

(21) Appl. No.: 17/043,255

(22) PCT Filed: Mar. 29, 2019
(86) PCT No.: PCT/US19/25025

§ 371 Date: Sep. 29, 2020

Related U.S. Application Data
(60) Provisional application No. 62/630,157, filed on Feb. 13, 2018.
(60) Provisional application No. 62/739,320, filed on Sep. 30, 2018.
(60) Provisional application No. 62/739,466, filed on Oct. 1, 2018.

(51) Int. Cl. A61K 38/13; A61K 47/34; A61K 9/00

(52) U.S. Cl. CPC A61K 38/13; A61K 9/0024; A61K 47/34

(57) ABSTRACT

A solid matrix sustained release ophthalmic formulation for topical delivery of the ophthalmic drug cyclosporine to the eye, medical devices, drug cores, drug inserts and drug delivery systems comprising the formulation, methods of manufacturing the formulation, medical devices and their methods thereof for delivering the ophthalmic drug for a treatment period are provided herein.

### CROSS-REFERENCE TO RELATED APPLICATIONS

This application claims the benefit of U.S. Provisional Patent Application Nos. 62/650,157, filed on 29 Mar. 2018; 62/739,320 filed 30 Sep. 2018; and, 62/739,466 filed on 1 Oct. 2018, the contents of which are each incorporated herein by reference in their entirety.

### FIELD OF THE INVENTION

This application pertains generally to sustained release formulations for topical delivery of ophthalmic drugs to the eye and their uses thereof for methods of treating keratoconjunctivitis sicca (dry eye syndrome).

### BACKGROUND OF THE INVENTION

FIGS. 1-2 illustrate example views of anatomical tissue structures associated with an eye 100. Certain of the anatomical tissue structures shown may be suitable for treatment using the various lacrimal implants and methods discussed herein. The eye 100 is a spherical structure including a wall having three layers: an outer sclera 102, a middle choroid layer 104 and an inner retina 106. The sclera 102 includes a tough fibrous coating that protects the inner layers. It is mostly white except for the transparent area at the front, commonly known as the cornea 108, which allows light to enter the eye 100.

The choroid layer 104, situated inside the sclera 102, contains many blood vessels and is modified at the front of the eye 100 as a pigmented iris 110. A biconvex lens 112 is situated just behind the pupil. A chamber 114 behind the lens 112 is filled with vitreous humor, a gelatinous substance. Anterior and posterior chambers 116 are situated between the cornea 108 and iris 110, respectively and filled with aqueous humor. At the back of the eye 100 is the light-detecting retina 106.

The cornea 108 is an optically transparent tissue that conveys images to the back of the eye 100. It includes a vascular tissue to which nutrients and oxygen are supplied via bathing with lacrimal fluid and aqueous humor as well as from blood vessels that line the junction between the cornea 108 and sclera 102. The cornea 108 includes a pathway for the permeation of drugs into the eye 100.

Turing to FIG. 2, other anatomical tissue structures associated with the eye 100 including the lacrimal drainage system, which includes a secretory system 230, a distributive system and an excretory system, are shown. The secretory system 230 comprises secretors that are stimulated by blinking and temperature change due to tear evaporation and reflex secretors that have an efferent parasympathetic nerve supply and secrete tears in response to physical or emotional stimulation. The distributive system includes the eyelids 202 and the tear meniscus around the lid edges of an open eye, which spread tears over the ocular surface by blinking, thus reducing dry areas from developing.

The excretory system of the lacrimal drainage system includes, in order of flow, drainage, the lacrimal puncta, the lacrimal canaliculi, the lacrimal sac 204 and the lacrimal duct 206. From the lacrimal duct 206, tears and other flowable materials drain into a passage of the nasolacrimal system. The lacrimal canaliculi include an upper (superior) lacrimal canaliculus 208 and a lower (inferior) lacrimal canaliculus 210, which respectively terminate in an upper 212 and lower 214 lacrimal punctum. The upper 212 and lower 214 punctum are slightly elevated at the medial end of a lid margin at the junction 216 of the ciliary and lacrimal portions near a conjunctival sac 218. The upper 212 and lower 214 punctum are generally round or slightly ovoid openings surrounded by a connective ring of tissue. Each of puncta 212, 214 leads into a vertical portion 220, 222 of their respective canaliculus before turning more horizontal at a canaliculus curvature 250 to join one another at the entrance of the lacrimal sac 204. The canaliculi 208, 210 are generally tubular in shape and lined by stratified squamous epithelium surrounded by elastic tissue, which permits them to be dilated. As shown, a lacrimal canaliculus ampulla 252 exists near an outer edge of each canaliculus curvature 250.

A variety of challenges face patients and physicians in the area of drug delivery, for example, ocular drug delivery. In particular, the repetitive nature of the therapies (multiple injections, instilling multiple eye drop regimens per day), the associated costs, and the lack of patient compliance may significantly impact the efficacy of the therapies available, leading to reduction in vision and many times blindness.

Patient compliance in taking the medications, for example, instilling the eye drops, can be erratic, and in some cases, patients may not follow the directed treatment regime. Lack of compliance can include, failure to instill the drops, ineffective technique (instilling less than required), excessive use of the drops (leading to systemic side effects) and use of non-prescribed drops or failure to follow the treatment regime requiring multiple types of drops. Many of the medications may require the patient to instill them up to 4 times a day.

A conventional method of drug delivery is by topical drop application to the eye's surface. Topical eye drops, though effective, can be inefficient. For instance, when an eye drop is instilled in an eye, it often overfills the conjunctival sac (i.e., the pocket between the eye and the associated lids) causing a substantial portion of the drop to be lost due to overflow of the lid margin and spillage onto the cheek. In addition, a large portion of the drop remaining on the ocular surface can be washed away into and through a lacrimal canaliculus, thereby diluting the concentration of the drug before it can treat the eye. Further, in some cases, topically applied medications have a peak ocular effect within about two hours, after which additional applications of the medications should be performed to maintain the therapeutic benefit.

To compound ocular management difficulty, subjects often do not use their eye drops as prescribed. Noncompliance rates by drop users of 25% and greater have been previously reported. This poor compliance can be due to, for example, forgetfulness or an initial stinging or burning sensation caused by the eye drop and experience by a subject. Instilling eye drops in one's own eye can be difficult, in part because of the normal reflex to protect the eye. Therefore, one or more drops may miss the eye. Older subjects may have additional problems instilling drops due to arthritis, unsteadiness, and decreased vision. Pediatric and psychiatric populations pose difficulties as well.

One promising approach to ocular drug delivery is to place an implant that releases a drug in tissue in or near the eye. However, providing a sustained release of a particular ophthalmic drug at a therapeutic dose over a desired period of time is challenging. Moreover, use of a lacrimal implant provides a limited volume in which to include the drug and a sustained release matrix, wherein elution of the drug must be both relatively constant and at a therapeutic dose over the desired time period.

In light of the above, it would be desirable to provide sustained release of certain ophthalmic drugs that overcome at least the above-mentioned shortcomings.

### SUMMARY OF THE INVENTION

Herein are provided sustained release formulations for the topical delivery of ophthalmic drugs to the eye, drug inserts and drug delivery systems comprising the formulation, methods of manufacturing the formulation, drug inserts and their methods thereof for delivering the ophthalmic drug for at least two weeks to the eye.

In embodiments are provided a sustained release ophthalmic formulation for topical delivery of an ophthalmic drug, wherein the formulation comprises a) at least one hydrophobic polymer; b) a nonionic surfactant; and, c) the ophthalmic drug, wherein the formulation does not comprise a hydrophilic polymer and the formulation is adapted to release the ophthalmic drug at therapeutically effective levels each day for a period of about two weeks to about 8 weeks.

In embodiments are provided a sustained release ophthalmic formulation for topical delivery of an ophthalmic drug, wherein the formulation comprises a) at least one hydrophobic polymer; b) a nonionic surfactant; and, c) the ophthalmic drug, wherein the formulation does not comprise a hydrophilic polymer and wherein the hydrophobic polymer is polycaprolactone and is present from about 12.5 to 47.5% (w/w), the nonionic surfactant is polysorbate 80 and is present from about 0 to 22.5% (w/w), and the ophthalmic drug is cyclosporine and is present from about 20 to 80% (w/w).

In embodiments are provided a sustained release ophthalmic formulation for topical delivery of an ophthalmic drug, wherein the formulation comprises a) at least one hydrophobic polymer; b) a nonionic surfactant; and, c) the ophthalmic drug, wherein the formulation does not comprise a hydrophilic polymer and wherein the hydrophobic polymer is polycaprolactone and is present from 15 to 30% (w/w), the nonionic surfactant is polysorbate 80 and is present from 4.5 to 10% (w/w), and the ophthalmic drug is cyclosporine and is present from 70 to 80% (w/w).

In embodiments are provided sustained release ophthalmic formulation for topical delivery of an ophthalmic drug, wherein the formulation comprises a) one or more hydrophobic polymers; and, b) the ophthalmic drug, wherein the formulation does not comprise a hydrophilic polymer or a nonionic surfactant and the formulation is adapted to release the ophthalmic drug at therapeutically effective levels each day for a period of about two weeks to about 8 weeks. In embodiments, a first hydrophobic polymer is polycaprolactone and is present from 15 to 30% (w/w), a second hydrophobic polymer is polyvinyl acetate and is present from 0 to 15% (w/w), and the ophthalmic drug is cyclosporine and is present from 70 to 80% (w/w).

In other embodiments are provided sustained release ophthalmic formulations for topical delivery of an ophthalmic drug, comprising cyclosporine admixed with one ore more hydrophobic polymers and optionally a nonionic surfactant to form a solid matrix composition, wherein the composition is in the form of a drug core and configured for placement within a lacrimal canaliculus.

In embodiments, the formulations are configured as a medical device including lacrimal implants, punctal plugs, intracanalicular plugs, or ocular rings. In embodiments, the formulations are configured for deposition within or adjacent to an eye. In certain embodiments, the medical device has a substantially cylindrical shape. In certain other embodiments, the medical device has a shape of a ring configured to be placed on a surface of an eye. In embodiments, the formulation further comprises a sheath body disposed at least partially over the matrix. In certain embodiments, the ophthalmic drug of the formulation is a powder, or weakly soluble in water.

In embodiments provided herein is a drug insert comprising a present sustained release formulation as a drug core and an impermeable sheath body partially covering the drug core. In embodiments, the drug insert is manufactured by extruding an admixture of drug and polymer (e.g. present sustained release formulation) into the impermeable sheath, optionally cut to a desirable length and optionally sealing one end. In embodiments the drug inserts are cut to a length of about 0.95 inches and one end sealed with a medical grade adhesive.

In embodiments, the present drug insert is placed in a cavity of a lacrimal implant to form a drug delivery system. In embodiments provided herein is a lacrimal implant comprising a punctal plug comprising a plug body and a drug insert, wherein the insert comprises; a drug core comprising the present sustained release formulation, and an impermeable sheath body partially covering the drug core, wherein the sheath body is configured to provide an exposed proximal end of the drug core in direct contact with tear fluid that releases therapeutic agent to the eye when the drug insert is disposed within a channel of the punctal plug and the punctal plug is inserted into the lacrimal canaliculus of a patient.

In embodiments provided herein, the sustained release formulation, as a medical device, drug insert or drug delivery system, is used to deliver an ophthalmic drug to an eye for treatment of dry eye. In embodiments provided herein is a method for delivering a drug for dry eye treatment to the eye, comprising, placing a lacrimal implant through a punctum and into a canalicular lumen of a patient, the implant comprising; a present sustained release ophthalmic formulation, wherein the ophthalmic drug is a cyclosporine and the matrix is configured for delivery of a daily therapeutic amount of cyclosporine for a period of at least 2 weeks and up to 6 months.

### BRIEF DESCRIPTION OF THE DRAWINGS

In the drawings, like numerals describe similar components throughout the several views. Like numerals having different letter suffixes represent different instances of similar components. The drawings illustrate generally, by way of example, but not by way of limitation, various embodiments disclosed herein.

FIG. 1 illustrates an example of anatomical tissue structures associated with an eye, certain of these tissue structures providing a suitable environment in which a lacrimal implant can be used.

FIG. 2 illustrates another example of anatomical tissue structures associated with an eye, certain of these tissue structures providing a suitable environment in which a lacrimal implant can be used.

FIG. 3A provides a perspective view of an implant in accordance with an embodiment of the present invention.

FIG. 3B is a side view of an implant in accordance with an embodiment of the present invention.

FIG. 3C is a side view illustrating the second member and the third member of an implant in accordance with an embodiment of the present invention.

FIG. 3D is a back view of an implant in accordance with an embodiment of the present invention.

FIG. 3E is a cross-sectional view taken about line III(E)-III(E) of FIG. 3D depicting an implant with a bore, in accordance with an embodiment of the present invention.

FIG. 3F is a partially enlarged view of FIG. 3E taken about circle III(F) depicting the second member, the third member and a bore formed in the third member of an implant, in accordance with an embodiment of the present invention.

FIG. 4A provides a perspective view of an implant in accordance with an embodiment of the present invention.

FIG. 4B is a cross-sectional view depicting an implant having a cavity formed in the second member, in accordance with an embodiment of the present invention.

FIG. 4C is a partially enlarged view taken about circle IV(C) of FIG. 4B depicting a cavity in the second member and a bore in the third member of an implant, in accordance with an embodiment of the present invention.

FIG. 5 provides a partial cross-sectional view of an implant in accordance with one embodiment of the present invention.

FIG. 6 provides a partial cross-section view of an implant in accordance with another embodiment of the present invention.

FIG. 7 shows elution data of cyclosporine from drug cores manufactured with polycaprolactone (PLC) at a range of 17.5 to 32.5% (w/w); polysorbate 80 (PS80) at a range of 7.5 to 22.5% (w/w); and, cyclosporine at a range of 60 to 67.5% (w/w) over a time period of 67 days. The different ratio of components in the formulations are presented as cyclosporine/Polysorbate 80/polycaprolactone in the Figure. The formulations all show an elution rate of at least 1.5 μg/day at day 48 of cyclosporine.

FIG. 8 shows the same elution data as FIG. 7, but over a shorter time period of 35 days.

FIG. 9 shows elution data of cyclosporine from drug cores manufactured with polycaprolactone (PLC) at a range of 14 to 25.5% (w/w); polysorbate 80 (PS80) at a range of 4.5 to 7.5% (w/w); and, cyclosporine at a range of 70 to 80% (w/w/) over a time period of 34 days. The different ratio of components in the formulations are presented as cyclosporine/PS80/PCL. The formulations all show an elution rate above the target (1.5 μg/day) for 34 days of cyclosporine.

FIG. 10 shows elution data of cyclosporine from drug cores of different lengths (950 μm to 1100 μm) manufactured with polycaprolactone (PLC) at about 30% (w/w); and, cyclosporine at about 70% (w/w) over a time period of 50 days. The formulations all show an elution rate of at least 1.5 μg/day at day 45 of cyclosporine.

FIG. 11 shows elution data of cyclosporine from drug cores manufactured with polycaprolactone (PLC) at a range of 15 to 25% (w/w); polyvinyl acetate at a range of 0 to 15% (w/w); and, cyclosporine at a range of 70 to 80% (w/w) over a time period of 60 days. The formulations all show an elution rate of at least 1.5 μg/day at day 55 of cyclosporine.

FIG. 12 shows elution data of cyclosporine from drug cores manufacture with polycaprolactone (PLC) at a range of 17 to 30% (w/w); polyvinyl acetate at a range of 0 to 5% (w/w); polysorbate 80 at a range of 0 to 3% (w/w) and, cyclosporine at a range of 70 to 75% (w/w) over a time period of 65 days. The formulations all show an elution rate of at least 1.5 μg/day at day 54 of cyclosporine.

FIG. 13 shows elution data of cyclosporine from drug cores with a length of 1100 μm manufactured with polycaprolactone (PLC) at about 17 or 20% (w/w); polyvinyl acetate at about 5% (w/w), polysorbate 80 at 0 or 3% (w/w), and, cyclosporine at about 75% (w/w) over a time period of 65 days. The formulations all show an elution rate of at least 1.5 μg/day at day 60 of cyclosporine.

FIG. 14 shows elution data of cyclosporine from drug cores manufacture with polycaprolactone (PLC) at a range of 5 to 20% (w/w); polyvinyl acetate at a range of 5 to 20% (w/w); no polysorbate 80; and, cyclosporine at 75% (w/w) over a time period of 13 days.

FIG. 15 shows elution data of cyclosporine from drug cores manufacture with polycaprolactone (PLC) at a range of 5 to 17% (w/w); polyvinyl acetate at a range of 5 to 17% (w/w); polysorbate 80 at 3% (w/w) and, cyclosporine at 75% (w/w) over a time period of 13 days.

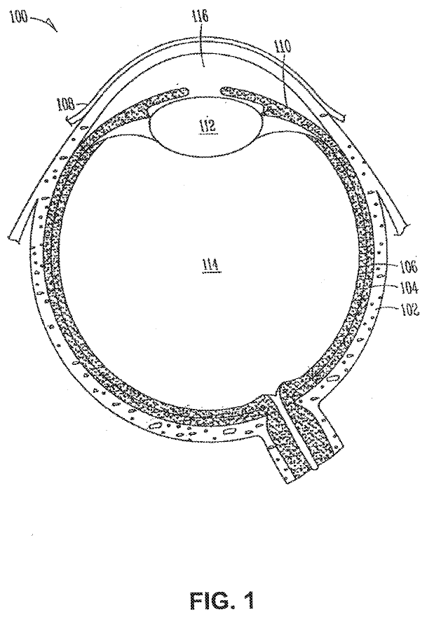

### DETAILED DESCRIPTION OF THE INVENTION

### Introduction

Provided herein are compositions, methods of manufacture and methods for the sustained topical delivery of an ophthalmic drug to an eye. In embodiments, the compositions comprise an ophthalmic drug (e.g., cyclosporine) admixed with one or more hydrophobic polymers and optionally a non-ionic surfactant to form a solid matrix composition, wherein the formulation does not comprise a hydrophilic polymer and the formulation is adapted to release the ophthalmic drug at therapeutically effective levels each day for a period of about two weeks to about 8 weeks. In certain embodiments, the compositions comprise an ophthalmic drug (e.g., cyclosporine) admixed with one or more hydrophobic polymers to form a solid matrix composition, wherein the formulation does not comprise a hydrophilic polymer or a non-ionic surfactant, and the formulation is adapted to release the ophthalmic drug at therapeutically effective levels each day for a period of about two weeks to about 8 weeks.

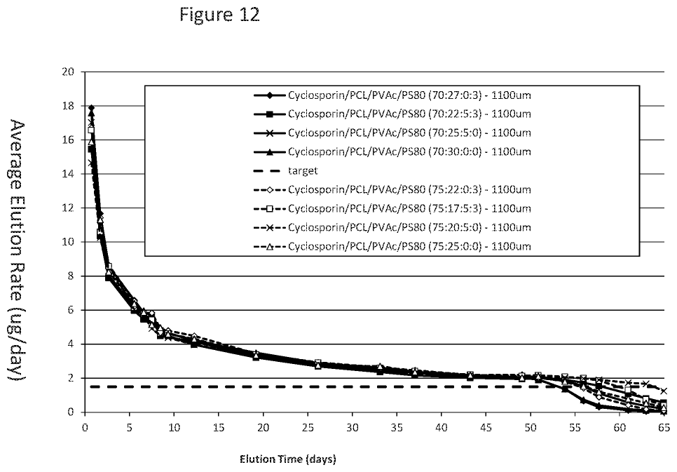

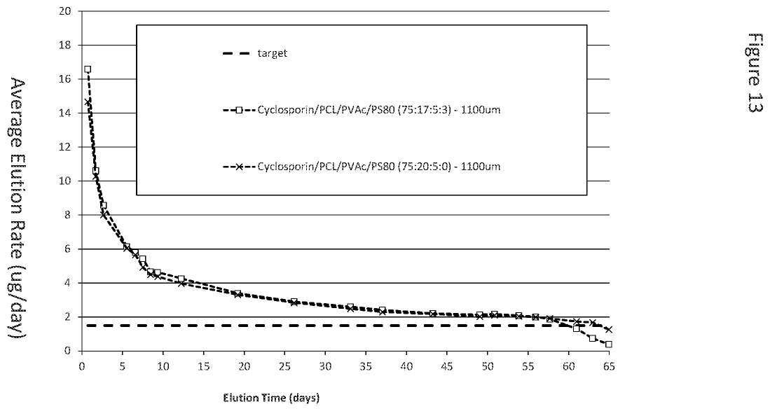

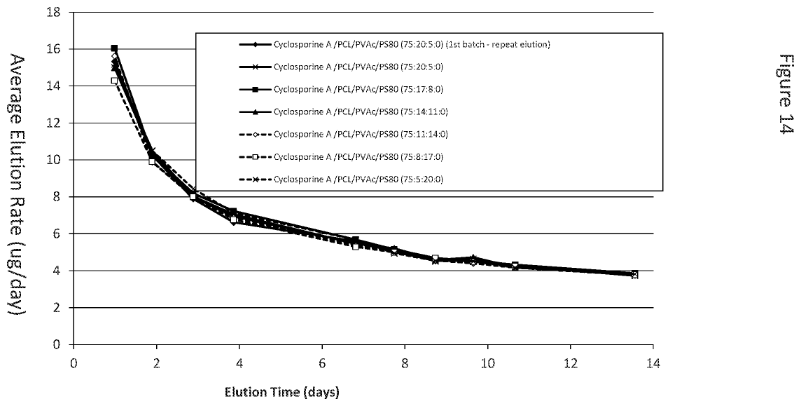

Without wishing to be bound by theory, the removal of hydrophilic polymers and/or nonionic surfactants increase the duration for elution (without negatively impacting an initial burst of drug) of a therapeutic dose (e.g. 1.5 μg/day of cyclosporine) of an ophthalmic drug without increasing the overall amount of drug present in the formulation. We have found that a formulation of cyclosporine admixed with a hydrophobic polymer, hydrophilic polymer and a nonionic surfactant demonstrates an elution of a therapeutic dose for up to about 35 days, while removing the hydrophilic polymer from that formulation increases the elution of drug at therapeutic levels to about 48 days (e.g. about 6 to 7 weeks). Removing the nonionic surfactant or including at a low amount (e.g. less than 5%(w/w)) from that formulation further increases the duration of elution of drug at therapeutic doses for up to 8 weeks. See FIGS. 12 and 13.

In other embodiments, the compositions comprise a sustained release formulation drug core comprising cyclosporine admixed with one ore more hydrophobic polymers and an optional nonionic surfactant to form a solid matrix composition, wherein the composition is in the form of a drug core and configured for placement within a lacrimal canaliculus. In certain embodiments, the solid matrix formulation and drug cores further comprise an impermeable sheath disposed at least partially over the solid matrix. The formulations were herein designed to topically deliver to the eye a daily therapeutic dose of cyclosporine for the treatment of dry eye.

Cyclosporine is an FDA approved drug, originally isolated from a fungus, indicated for the treatment of signs and symptoms of dry eye, a syndrome called keratoconjunctivitis sicca. Cyclosporine is an immunosuppressive drug and reduces inflammation including reducing activity of T cells in the conjunctiva tissue of the eye.

### DEFINITIONS

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, an “axis” refers to a general direction along which a member extends. According to this definition, the member is not required to be entirely or partially symmetric with respect to the axis or to be straight along the direction of the axis. Thus, in the context of this definition, any member disclosed in the present application characterized by an axis is not limited to a symmetric or a straight structure.

In this document, the term “proximal” refers to a location relatively closer to the cornea of an eye, and the term “distal” refers to a location relatively further from the cornea and inserted deeper into a lacrimal canaliculus.

In the appended claims, the terms “including” and “in which” are used as the plain-English equivalents of the respective terms “comprising” and “wherein.” Also, in the following claims, the terms “including” and “comprising” are open-ended, that is, a system, assembly, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

### Compositions

In embodiments, the composition comprises the present sustained release formulation as a medical device, as a drug core, as a drug insert (e.g. present formulation and an outer layer or covering), and as a drug delivery system (e.g. drug insert or core and a body or retention element to maintain the drug insert or core in a desired location). In embodiments, the medical device (e.g. drug core or drug insert) may be placed in the lacrimal canaliculus or between a sclera tissue layer, such as between the surface of the eye and eye lid (e.g. an ocular ring placed outside the field of vision), or between a sclera tissue layer and a conjunctiva tissue layer of the eye to deliver the ophthalmic drug to the eye. In embodiments, the medical device comprises a substantially cylindrical diameter over the length of the medical device and may be configured for either placement in a lacrimal canaliculus (e.g. intracanalicular plug) or between an eyelid and the surface of the eye, which may be in the shape of a ring or linear. In alternative embodiments, the drug insert is adapted to be placed in a body of the drug delivery system. The ocular drug delivery system, disclosed in more detail below, uses a body that is interchangeable with a drug insert and/or drug core comprising different drugs and/or different matrix to provide topical sustained release of the drug.

In embodiments, the lacrimal implant of the invention is configured as a sustained release device, releasing the incorporated ophthalmic drug (e.g., cyclosporine) in a therapeutically effective manner, e.g., at a rate that provides a therapeutically effective dosage for at least about 1 week, 2 weeks, 3 weeks, 4 weeks, 5 weeks, 6 weeks, 7 weeks, 8, weeks, 9 weeks 10 weeks, 11 weeks, or at least about 12 weeks or more. For cyclosporine, a therapeutic level is an average daily elution rate of at least 1.5 μg/day of the drug. In an exemplary embodiment, the lacrimal implant is configured to be retained by the puncta for the duration of the intended controlled release of the therapeutic agent. In various embodiments, the duration of the intended controlled release of the therapeutic agent is at least about 1 week, 2 weeks, 3 weeks, 4 weeks, 5 weeks, 6 weeks, 7 weeks, 8, weeks, 9 weeks 10 weeks, 11 weeks, or at least about 12 weeks or more. In various embodiments at least 95% of the implanted implants are retained for the duration of the intended controlled release of the therapeutic agent. In an exemplary embodiment, the implant is retained by the puncta for a length of time to show therapeutic efficacy.

In embodiments, the present solid matrix sustained release ophthalmic formulations comprise from about 20% to about 80% w/w, from about 30% to about 80% w/w, from about 40% to about 80% w/w, from about 50% to about 80% w/w, from about 60% to about 80% w/w, or from about 70% to about 80% w/w of the ophthalmic drug. In embodiments, the ophthalmic drug is cyclosporine. In exemplary embodiments, the present solid matrix sustained release ophthalmic formulations comprise from about 60% to about 80% w/w of cyclosporine. In other exemplary embodiments, the present solid matrix sustained release ophthalmic formulations comprise from about 70% to about 80% w/w of cyclosporine. In certain embodiments, the present solid matrix sustained release ophthalmic formulations comprise about 75% w/w of cyclosporine.

In embodiments, the present solid matrix sustained release ophthalmic formulations comprise from about 20 to about 80% (w/w), from about 20 to about 75% (w/w), from about 20 to about 70% (w/w), from about 20 to about 65% (w/w), from about 20% to about 60% w/w, from about 20% to about 55% w/w, from about 20% to about 50% w/w, from about 20 to about 45% (w/w), from about 20 to about 40% (w/w), from about 20 to about 35% (w/w), or from about 20% to about 30% w/w of the ophthalmic drug. In other embodiments, the present solid matrix sustained release ophthalmic formulations comprise from about 50% to about 80% w/w, to about 55% to about 80% w/w, from about 60% to about 80% w/w, from about 65% to about 80% w/w, or from about 70% to about 80% w/w of the ophthalmic drug. In certain other embodiments, the present solid matrix sustained release ophthalmic formulations comprise about 55%, about 57.5%, about 60%, about 62.5%, about 65%, about 67.5%, about 70%, about 72.5%, about 75%, about 77.5%, about 80%, about 82.5%, or about 85% w/w of the ophthalmic drug. In embodiments, the ophthalmic drug is cyclosporine.

In certain embodiments, the present solid matrix sustained release ophthalmic formulations comprise from about 70% to about 80% w/w of an ophthalmic drug. In embodiments, the ophthalmic drug is present in the solid matrix formulation at about 70%, about 71%, about 72%, about 73%, about 74%, about 75%, about 76%, about 77%, about 78%, about 79%, or about 80% (w/w). The % numbers are inclusive of 0.5% above and below each of the whole percentage numbers, providing a range for “about”. For example, about 75% is inclusive of 74.5, 74.75, 75, 75.25, 75.50 and each value in between thereof.

In certain embodiments, the present solid matrix sustained release ophthalmic formulations further comprises one or more hydrophobic polymers and optionally a nonionic surfactant. In exemplary embodiments, the ophthalmic drug is cyclosporine.

In embodiments, the present sustained release ophthalmic formulations comprise about 60 to about 240 μg of cyclosporine.

In embodiments, the solid matrix sustained release ophthalmic formulation for topical delivery of the ophthalmic drugs disclosed above are used for the treatment of dry eye. In embodiments, a therapeutic dose of cyclosporine, as eluted from the present sustained release formulation when placed in or around the eye, is about 1.5 μg to about 3 μg of cyclosporine a day.

In embodiments, the formulation is prepared by dissolving the drug, polymer mixture and optional nonionic surfactant and then forming into a desired shape. In embodiments, the formulation is extruded into a sheath body to form a drug insert, which may be used with a lacrimal implant or device (e.g. drug delivery system). In other embodiments, the drug core or drug insert does not comprise an impermeable sheath body or other permeable layer distinct from the solid sustained release formulation matrix.

### Sustained Release Formulation Components

In embodiments, the present sustained release ophthalmic formulations comprise one or more hydrophobic polymers. The term “hydrophobic” as used herein is generally understood to be a polymer that has a limited affinity for water and does not mix well with water. For example, hydrophobic polymers may be non-polar and will aggregate in an aqueous solution and exclude water molecules. The exclusion of water maximizes the hydrogen bonding of the hydrophobic polymer, either to other hydrophobic polymers, a hydrophilic polymer or possibly even a surfactant. In embodiments, hydrophobic polymers include for example, non-polar polymers, polyester polymers, PLGA, PLA, polycaprolactone, and polyanhydrides with hydrophobic co-monomer (e.g. carboxyphenoxypropane). In certain embodiments, the hydrophobic polymer is selected from polyester, polycaprolactone, polyvinyl acetate (PVAc), poly(D,L-lactic-co-glycolic acid) (PLGA), poly lactic acid (PLA), polyurethane, poly glycolic acid (PGA) or a combination thereof. In certain embodiments, the hydrophobic polymer comprises silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate), poly(vinyl alcohol) (PVA), poly(vinyl ether), poly(vinyl ketone), poly(vinylpyrrolidone (PVP), poly(vinylpyridine), co-polymers thereof, or combinations thereof. In embodiments, the present solid matrix sustained release ophthalmic formulations comprise polycaprolactone as the one or more hydrophobic polymers. In certain exemplary embodiments, the present solid matrix sustained release ophthalmic formulations comprise polycaprolactone and polyvinyl acetate as a first and second hydrophobic polymer of the one or more hydrophobic polymers.

In embodiments, the present solid matrix sustained release ophthalmic formulations comprise from about 10% to about 50% w/w, from about 20% to about 50% w/w, from about 30% to about 50% w/w, or from about 40% to about 50% w/w of the hydrophobic polymer. In embodiments, the one or more hydrophobic polymer is polycaprolactone. In exemplary embodiments, the present solid matrix sustained release ophthalmic formulations comprise from about 5% to about 32% w/w of polycaprolactone. In other exemplary embodiments, the present solid matrix sustained release ophthalmic formulations comprise from about 15% to about 30% w/w of polycaprolactone.

In exemplary embodiments, the hydrophobic polymer is polyvinyl acetate. In exemplary embodiments, the present solid matrix sustained release ophthalmic formulations comprise from about 0% to about 20% w/w of polyvinyl acetate. In other exemplary embodiments, the present solid matrix sustained release ophthalmic formulations comprise from about 5% to about 20% w/w of polyvinyl acetate.

In embodiments, the present solid matrix sustained release ophthalmic formulations comprise from about 10 to about 50% (w/w), from about 10 to about 45% (w/w), from about 10 to about 40% (w/w), from about 10 to about 35% (w/w), from about 10% to about 30% w/w, from about 10% to about 25% w/w, from about 10% to about 20% w/w, or from about 10 to about 15% (w/w) of the one or more hydrophobic polymers. In certain embodiments, the combined total of one or more hydrophobic polymers are not more than 30% w/w of the total formulation.

In certain embodiments, the present solid matrix sustained release ophthalmic formulations comprise from about 10% to about 35% w/w of one or more hydrophobic polymers. In embodiments, the one or more hydrophobic polymers are present in the solid matrix formulation at about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34% or about 35% (w/w) of the total hydrophobic polymer. The % numbers are inclusive of 0.5% above and below each of the whole percentage numbers, providing a range for “about”. For example, about 20% is inclusive of 19.5, 19.75, 20, 20.25, 20.50 and each value in between thereof. In certain embodiments, the present solid matrix sustained release ophthalmic formulations further comprise cyclosporine. In exemplary embodiments, the hydrophobic polymer is polycaprolactone. In other exemplary embodiments, the one or more hydrophobic polymers comprise polycaprolactone and polyvinyl acetate.

In embodiments, the present solid matrix sustained release ophthalmic formulations do not comprise silicone. In certain embodiments, the present solid matrix sustained release ophthalmic formulations do not comprise methacrylate polymers or monomers.

In embodiments, the present solid matrix sustained release ophthalmic formulations do not comprise a hydrophilic polymer. As used herein, the term “hydrophilic” is understood to be a polymer that has a strong affinity for water and may be readily soluble in water. For example, hydrophilic polymers may be polar and their interaction with water (and other polar) substances are more thermodynamically favorable than interactions with hydrophobic polymers or substances. In embodiments, hydrophilic polymers excluded from the present solid matrix sustained release ophthalmic formulations include for example, polar polymers, polysaccharides including alginate and chitosan, hydrophilic polyanhydrides, polyethylene glycol (PEG), proteins, DNA, and polyvinyl alcohol. In certain embodiments, the excluded hydrophilic polymers include polyethylene glycol (PEG) polymers, acrylate-derivatized PEG (PEGDA) polymers, polysaccharide polymers, hydrophilic polyanhydrides or a combination thereof. In certain embodiments, the excluded hydrophilic polymers include polyethylenimine (PEI), poly(ethylene glycol) (PEG), poly(oxyethylene), poly(ethylene oxide) (PEO), poly(acrylic acid) (PAA), poly(methacrylic acid), poly(vinyl alcohol) (PVA), poly(vinylpyrrolidone (PVP), polyelectrolytes, poly(maleic anhydride acid), poly(ether), acrylate-derivatized PEG (PEGDA) polymers, polysaccharide polymers, hydrophilic polyanhydrides, co-polymers thereof, or combinations thereof. In certain embodiments, the present solid matrix sustained release ophthalmic formulations do not comprise polyethylene glycol (PEG) polymers, acrylate-derivatized PEG (PEGDA) polymers, polysaccharide polymers, hydrophilic polyanhydrides or a combination thereof.

In embodiments, the present solid matrix sustained release ophthalmic formulations comprise a nonionic surfactant. In alternative embodiments, the present solid matrix sustained release ophthalmic formulations do not comprise a nonionic surfactant. As used herein “surfactant” refers to a compound that lowers the surface tension between two liquids or between a liquid and a solid. Surfactants are typically amphiphilic, meaning they comprise both a hydrophilic moiety and a hydrophobic moiety, such as fatty alcohol groups and compounds that form micelles in an aqueous solution. Nonionic surfactants have covalently bonded oxygen-containing hydrophilic groups, which are bonded to hydrophobic parent structures; an amphiphilic compound. The water-solubility of the oxygen groups is the result of hydrogen bonding. The differences between the individual types of nonionic surfactants are slight, and the choice is primarily governed based on the cost of special properties, e.g., effectiveness and efficiency, toxicity, dermatological compatibility and biodegradability, or permission for use in pharmaceutical products. In the instant solid matrix sustained release ophthalmic formulations, the choice of an individual surfactant may also be governed by improved efficiency in manufacturing, e.g. extrusion of the formulation into a mold or tubing, such as a sheath body. For example, use of tyloxapol or polysorbate may provide little difference in daily elution rate, however one may provide for improved extrusion during manufacturing depending on the choice of hydrophobic polymers and their overall % w/w in the matrix. In certain exemplary embodiments, the present solid matrix sustained release ophthalmic formulations comprise a polysorbate surfactant such as polysorbate 80.

In exemplary embodiments, the present solid matrix sustained release ophthalmic formulations do not comprise hydrophilic polymers or amphiphilic polymers or molecules.

Examples of nonionic surfactants include fatty alcohol ethoxylates, alkylphenol ethoxylates, fatty acid ethoxylates (e.g. polysorbate), certain ethoxylated fatty esters and oils, ethoxylated amines and/or fatty acid amides, terminally blocked ethoxylates, fatty acid esters of polyhydroxy compounds, fatty acid esters of glycerol, fatty acid esters of sorbitol (e.g. Spans), fatty acid esters of sucrose, alkyl polyglucosides, amine oxides, sulfoxides, polymers of alkyl aryl polyether alcohol (e.g. tyloxapol), polyoxyethylene ethers (e.g. BRIJ compounds) and phosphine oxides.

Polysorbate surfactants are ethoxylated sorbitan esters and include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate), wherein the number 20 following the ‘polyoxyethylene’ part refers to the total number of oxyethylene —(CH₂CH₂O)— groups found in the molecule. The number following the ‘polysorbate’ part is related to the type of fatty acid associated with the polyoxyethylene sorbitan part of the molecule. Monolaurate is indicated by 20, monopalmitate is indicated by 40, monostearate by 60, and monooleate by 80. In exemplary embodiments, the present solid matrix sustained release ophthalmic formulations comprise the nonionic surfactant polysorbate 80.

BRIJ nonionic surfactants are polyoxyethylene ethers and include, polyoxyethylene (20) oleyl ether, polyoxyethylene (10) oleyl ether, polyoxyethylene (2) oleyl ether, polyoxyethylene (100) stearyl ether, polyoxyethylene (20) cetyl ether, polyoxyethylene (10) cetyl ether, polyoxyethylene (10) stearyl ether, polyoxyethylene (4) lauryl ether, polyoxyethylene (20) stearyl ether, polyoxyethylene (2) cetyl ether, and polyoxyethylene (2) stearyl ether.

Span nonionic surfactants are sorbitan esters that include sorbitan oleate, sorbitan stearate, sorbitan laurate, sorbitane trioleate, sorbitan tristearate, sorbitan sesquioleate, and sorbitan monopalmitate. In embodiments, the present solid matrix sustained release ophthalmic formulations comprise the nonionic surfactant sorbitan ester. In certain embodiments, the present solid matrix sustained release ophthalmic formulations comprise a combination of the nonionic surfactants sorbitan ester (e.g. Span 40) and polysorbate (e.g. polysorbate 80). In certain embodiments, the present solid matrix sustained release ophthalmic formulations comprise a combination of the nonionic surfactants sorbitan ester (e.g. Span 40) and polysorbate (e.g. polysorbate 80), wherein the solid matrix does not comprise a hydrophilic polymer as disclosed above.

In certain embodiments, the present solid matrix sustained release ophthalmic formulations comprise from about 1% to about 10% w/w of a nonionic surfactant. In embodiments, the nonionic surfactant is present in the solid matrix formulation at about 1%, about 2%, about 3%, about 4%, about 5%, about 6%, about 7%, about 8%, about 9% or about 10% (w/w). The % numbers are inclusive of 0.5% above and below each of the whole percentage numbers, providing a range for “about”. For example, about 4% is inclusive of 3.5, 3.75, 4, 4.25, 4.50 and each value in between thereof. In certain embodiments, the present solid matrix sustained release ophthalmic formulations further comprise polycaprolactone. In exemplary embodiments, the nonionic surfactant is a polysorbate.

In certain embodiments, the present solid matrix sustained release ophthalmic formulations comprise from about 0% to about 25% w/w of a nonionic surfactant. In embodiments, the nonionic surfactant is present in the solid matrix formulation at about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% (w/w). The % numbers are inclusive of 0.5% above and below each of the whole percentage numbers, providing a range for “about”. For example, about 20% is inclusive of 19.5, 19.75, 20, 20.25, 20.50 and each value in between thereof. In certain embodiments, the present solid matrix sustained release ophthalmic formulations further comprise polycaprolactone. In exemplary embodiments, the nonionic surfactant is a polysorbate.

In certain embodiments, the present solid matrix sustained release ophthalmic formulations comprise a nonionic surfactant selected from tyloxapol, sorbitan esters, polyoxyethylene ethers, a polysorbate or a combination thereof.

In embodiments, the present sustained release ophthalmic formulations comprise a) one or more hydrophobic polymers; b) an optional nonionic surfactant; and, c) the ophthalmic drug, wherein the formulation does not comprise a hydrophilic polymer and wherein the hydrophobic polymer is polycaprolactone and is present from about 5 to 47.5% (w/w), the nonionic surfactant is polysorbate 80 and is present from about 0 to 22.5% (w/w), and the ophthalmic drug is cyclosporine and is present from about 20 to 80% (w/w).

In embodiments, the present solid matrix sustained release ophthalmic formulations comprise one or more hydrophobic polymers; an optional nonionic surfactant and ophthalmic drug, wherein the hydrophobic polymer is polycaprolactone and is present from 5 to 30% (w/w), the nonionic surfactant is polysorbate 80 and is present from 0 to 10% (w/w), and the ophthalmic drug is cyclosporine and is present from 70 to 80% (w/w).

In embodiments, the present solid matrix sustained release ophthalmic formulations comprise a) one or more hydrophobic polymers; and, b) the ophthalmic drug, wherein the formulation does not comprise a hydrophilic polymer or a nonionic surfactant, wherein a first hydrophobic polymer is polycaprolactone and is present from 15 to 30% (w/w), a second hydrophobic polymer is polyvinyl acetate and is present from 0 to 15% (w/w), and the ophthalmic drug is cyclosporine and is present from 70 to 80% (w/w).

In embodiments, the present solid matrix sustained release ophthalmic formulations comprise a) one or more hydrophobic polymers; b) an optional nonionic surfactant; and, c) the ophthalmic drug, wherein the formulation does not comprise a hydrophilic polymer, wherein a first hydrophobic polymer is polycaprolactone and is present from 5 to 30% (w/w), a second hydrophobic polymer is polyvinyl acetate and is present from 5 to 20% (w/w), the nonionic surfactant is polysorbate 80 and is present from 0 to 5% w/w, and the ophthalmic drug is cyclosporine and is present from 70 to 80% (w/w).

In certain embodiments, an impermeable sheath body (disclosed in more detail below) is disposed over at least a portion of the solid matrix composition.

### Lacrimal Implants

In embodiments, provided herein are lacrimal implants comprising a punctal plug comprising a plug body and a drug insert, wherein the insert comprises; a drug core comprising any one of the present solid matrix sustained release ophthalmic formulations disclosed herein; and, an impermeable sheath body partially covering the drug core, wherein the sheath body is configured to provide an exposed proximal end of the drug core in direct contact with tear fluid that releases an ophthalmic drug to the eye when the drug insert is disposed within a channel of the punctal plug and the punctal plug is inserted into the lacrimal canaliculus of a patient.

In certain embodiments, the any one of the present solid matrix sustained release ophthalmic formulations disclosed herein are configured as a medical device for the delivery of the ophthalmic drug to the eye. Those medical devices may take the shape of a depot, a lacrimal implant with a separate body, an intracanalicular plug that does not further comprise a separate plug body or a sheath body, an ocular ring (such as one that is placed on the eye surface but under the eye lid), or a contact lens. In certain embodiments, the intracanalicular plug comprises a polymeric coating or layer completely or partially surrounding the plug. In embodiments, the medical device may comprise a coating or an internal filament to provide structural integrity to the medical device. In embodiments, the medical device has a substantially cylindrical shape wherein the diameter of the entire medical device is approximately the same at the time of placement in, on or near the eye.

In certain embodiments, the compositions of the invention comprise an implant including a distinct solid matrix formulation drug core or integrated drug or other agent disposed in at least one of the first member 305 or the second member 310 of the implant body, to provide a sustained release of a therapeutic agent (used interchangeably herein with ophthalmic drug). For instance, the drug core or integrated drug or other agent disposed may be disposed in the cavity 458 of the lacrimal implant 400 to provide a sustained drug or other therapeutic agent release.

An exemplary implant of use in the methods of the invention is configured to deliver a therapeutic agent to one or more of an eye, nasal passage or inner ear system. In various embodiments, the drug is delivered systemically to the subject through the eye. A therapeutic agent core can comprise one or more therapeutic agents, and in some examples, one or more matrix materials to provide sustained release of the drug or other agents.

In various embodiments, the drug core (used interchangeably herein with the present solid matrix sustained release ophthalmic formulation) is inserted into cavity 458.

In embodiments, the compositions comprise a drug insert comprising a sheath body and a present sustained release ophthalmic formulation. The sheath body can comprise appropriate shapes and materials to control the migration of ophthalmic drug from the drug core. In some embodiments, the sheath body houses the drug core and can fit snugly against the core. The sheath body is made from a material that is substantially impermeable to the anti-inflammatory agent so that the rate of migration of the agent may be largely controlled by the exposed surface area of the drug core that is not covered by the sheath body. In many embodiments, migration of the ophthalmic drug through the sheath body can be about one tenth of the migration of ophthalmic drug through the exposed surface of the drug core, or less, often being one hundredth or less. In other words, the migration of the ophthalmic drug through the sheath body is at least about an order of magnitude less that the migration of anti-inflammatory agent through the exposed surface of the drug core. Suitable sheath body materials include polyimide, polyethylene terephthalate (hereinafter “PET”). The sheath body has a thickness, as defined from the sheath surface adjacent the core to the opposing sheath surface away from the core, from about 0.00025″ to about 0.0015″. The total diameter of the sheath that extends across the core ranges from about 0.2 mm to about 1.2 mm. In embodiments, the drug core has a diameter from about 0.55 to about 0.70 mm. In certain embodiments, the drug core has a diameter of about 0.61 mm. The core may be formed by dip coating the core in the sheath material. Alternatively, or in combination, the sheath body can comprise a tube and the core introduced into the sheath, for example as a liquid or solid that can be slid, injected or extruded into the sheath body tube. The sheath body can also be dip coated around the core, for example dip coated around a pre-formed core.

It is generally understood that when the present solid matrix formulation is at least partially surrounded by a sheath body, the hydrophobic polymers do not erode. In other words, they are not biodegradable via hydrolysis or oxidation, even when those polymers may be biodegradable under different conditions (e.g., when not protected by a sheath body). Hence, while hydrophilic moieties present in the polymers and/or surfactants of the present solid matrix sustained release ophthalmic formulations may bind water molecules, such as present in tear fluid, the polymers do not generally undergo hydrolysis during the treatment period.

The sheath body can be provided with additional features to facilitate clinical use of the implant. For example, the sheath may receive a drug core that is exchangeable while the implant body, retention structure and sheath body remain implanted in the subject. The sheath body is often rigidly attached to the retention structure as described above, and the core is exchangeable while the retention structure retains the sheath body. In specific embodiments, the sheath body can be provided with external protrusions that apply force to the sheath body when squeezed and eject the core from the sheath body. Another drug core can then be positioned in the sheath body. In many embodiments, the sheath body or retention structure may have a distinguishing feature, for example a distinguishing color, to show placement such that the placement of the sheath body or retention structure in the canaliculus or other body tissue structure can be readily detected by the subject. The retention element or sheath body may comprise at least one mark to indicate the depth of placement in the canaliculus such that the retention element or sheath body can be positioned to a desired depth in the canaliculus based on the at least one mark.

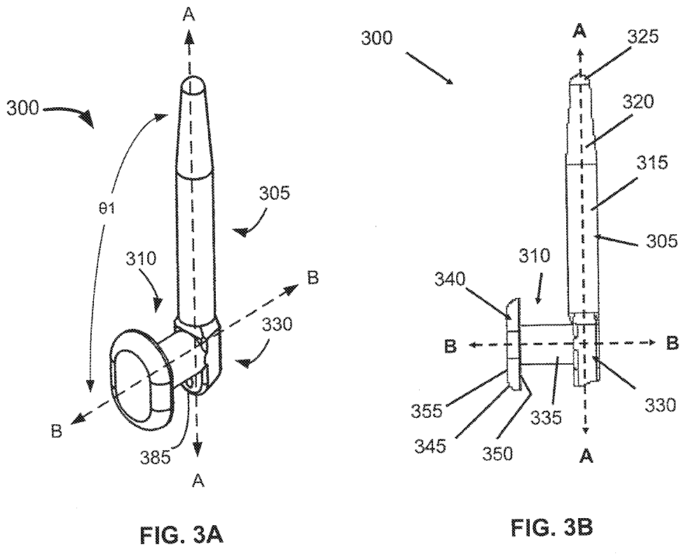

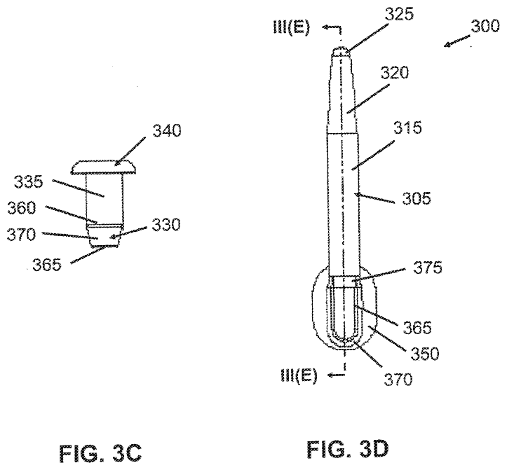

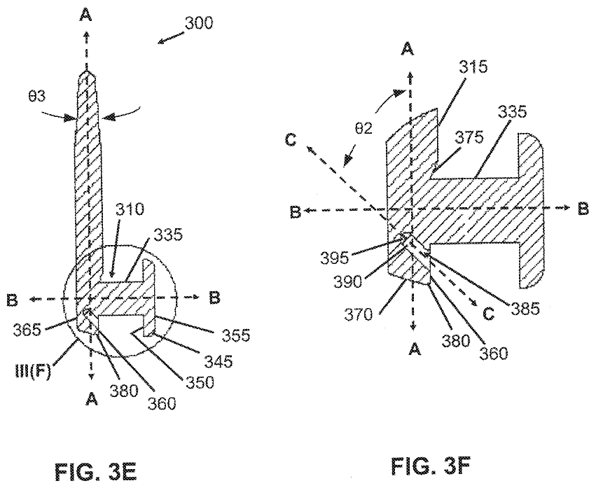

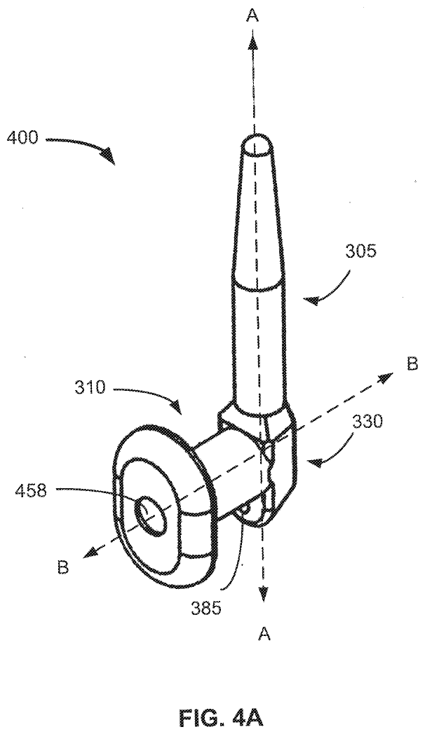

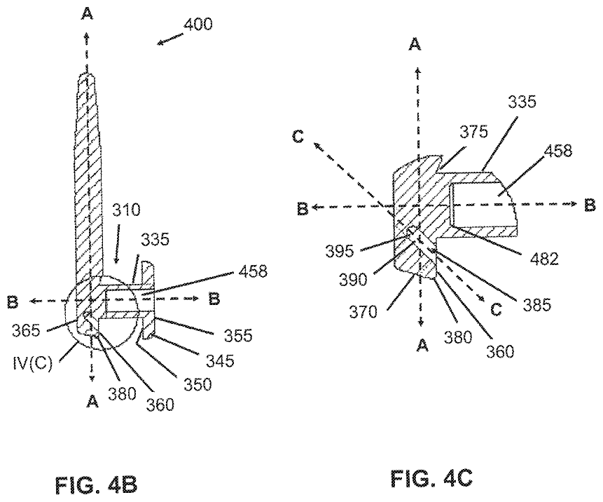

FIGS. 3-6 illustrate exemplary embodiments of lacrimal implants of use with the present formulations and in methods of the invention. The exemplary implants are insertable through a lacrimal punctum 212, 214 and into its associated canaliculus 208, 210. Exemplary lacrimal implants of use in the present invention comprise a first member, a second member and a heel, such as the first member 305, the second member 310 and the third member or heel 330 depicted in FIG. 3A. Exemplary lacrimal implants further comprise a bore that is formed in the heel, for example, the bore 385 formed in the third member or heel 330 in FIG. 3A. In some embodiments, exemplary lacrimal implants further comprise a cavity 458 (e.g., lacrimal implants illustrated in FIG. 4A).

Referring to FIG. 3A, where a perspective view of an exemplary lacrimal implant 300 of use in the present methods is depicted, the first member 305 is characterized by a first axis A and the second member 310 is characterized by a second axis B.

The third member or heel 330 is configured to connect the first member 305 and the second member 310 at a first angle θ₁, where θ₁ is defined by the first axis A with respect to the second axis B. For instance, in FIG. 3A, the first angle θ₁ refers to the angle originating at the first axis A and turning counterclockwise from the first axis A to the second axis B. In some embodiments, the first axis A and the second axis B are in the same plane and intersect each other. In some embodiments, the first axis A is in a plane other than the plane of the second axis B, and the first axis A and the second axis B do not intersect. In such embodiments, the first angle θ₁ refers to the angle defined by a parallel line of the first axis A with respect to the second axis B. This parallel line of the first axis A lies in the same plane as the second axis and intersects with the second axis.

In some embodiments, the first angle θ₁ is from about 30 degrees to about 150 degrees, from about 45 degrees to about 135 degrees, or from about 75 degrees to about 105 degrees. For example, in some embodiments, the first angle θ₁ is approximately 90 degrees.

In some embodiments, the overall dimension of the implant along the first axis is from about 4 mm to about 8 mm. In an exemplary embodiment, the overall dimension along the first axis is about 5 mm to about 7 mm. In various embodiments, the overall dimension along the first axis is about 6.3 mm.

In various embodiments, the overall dimension along the second axis B is from about 1 mm to about 3 mm, e.g., from about 1.2 mm to about 1.9 mm.

In some embodiments, the overall dimension along the first axis is approximately 6.3 mm and the overall dimension along the second axis is approximately 1.2 mm. In various embodiments, the overall dimension along the first axis is approximately 6.3 mm and the overall dimension along the second axis is approximately 1.9 mm. In some embodiments, the overall dimension along the first axis is approximately 4.8 mm and the overall dimension along the second axis is approximately 1.9 mm.

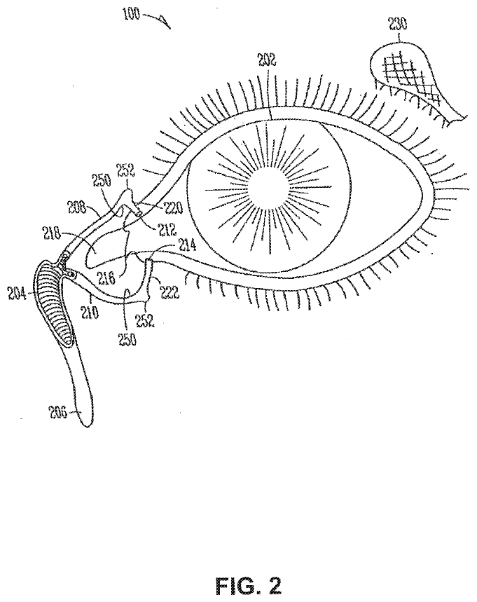

In some embodiments, the first member 305 is configured to extend into a canaliculus, while the second member 310 is configured to reside in the vertical portion 220, 222 of the canaliculus and to extend to the opening of, or out of the opening of, the associated puncta. When a lacrimal implant 300 of such configuration is inserted into a canaliculus, the intersection of the first axis A and the second axis B resides generally at a curvature of the canaliculus, such as the canaliculus curvature 250 in FIG. 2. In some embodiments, the first member 305 and the second member 310 are connected at the first angle, and that angle is at least about 45 degree, thereby forming an angled intersection between the first member and the second member. In various embodiments, when the lacrimal implant 300 is positioned in the lacrimal canaliculus, at least a portion of the angled intersection is biased against a canaliculus curvature of the lacrimal canaliculus. In this embodiment, the lacrimal implant 300 uses anatomical structures to facilitate the retention of the implanted lacrimal implant 300.

FIG. 3B depicts a side view of an exemplary lacrimal implant 300 of the invention. In some embodiments, the first member 305 includes an intermediate segment 315, a tip segment or tip 325, and a forward segment 320 in between the forward segment and tip segment. While the intermediate segment 315 is configured to be connected to the second member 310 by the third member or heel 330, the tip segment or tip 325 is configured to be inserted through a punctum prior to the other two segments of the first member 305 and prior to the other members of the lacrimal implant 300.

In some embodiments, the intermediate segment 315, the forward segment 320 and the tip segment or tip 325 are distinguishable from each other in general by their shapes. For example, in some embodiments, the intermediate segment 315 has a generally cylindrical shape with a diameter that is larger than the diameter of the tip segment or tip 325. In various embodiments, the forward segment 320 is tapered and has a conical shape, such that the forward segment 320 connects the intermediate segment 315 at one end and the tip segment or tip 325 at the other end. In some embodiments, the transition from the intermediate segment 315 to the forward segment 320 or the transition from the forward segment 320 to the tip segment or tip 325 is gradual and smooth such that no distinguishable edge exists at the transition.

In some embodiments, the intermediate segment 315 has a cylindrical shape. In various embodiments, the intermediate segment has a circular cross section, an elliptic cross section, or a polygonal cross section. The intermediate segment 315 is of any useful combination of length and diameter.

In some embodiments, the intermediate segment 315 has a diameter that is from about 0.4 mm to about 0.8 mm. For example, in some embodiments the diameter of the intermediate segment 315 is from about 0.53 mm to about 0.63 mm. In some embodiments, the intermediate segment 315 has a length along the first axis A that is from about 0.5 mm to about 3.5 mm. For example, in some embodiments the length of the intermediate segment 315 is from about 1 mm to about 2.8 mm.

In some embodiments, the tip segment or tip 325 is substantially a semi-sphere, or a portion of a semi-sphere. In exemplary embodiments, the semi-sphere, or portion therapy, has a radius that is from about 0.05 mm to about 0.3 mm. For example, in some embodiments, the radius of the tip segment or tip 325 is approximately 0.20 mm.

In some embodiments, the forward segment 320 has a conical configuration, tapering from the diameter of the intermediate segment 315 as it approaches the tip segment or tip 325. In some embodiments, the forward segment 320 is short and is tapered steeply, thus forming a wider taper angle. The forward segment 320 can also be long and tapered more gradually, thus forming a narrower taper angle. The tapering angle θ₃ is illustrated in FIG. 3E. In some embodiments, the tapering angle θ₃ is from about 2° to about 10°. For example, in some embodiments the tapering angle θ₃ is from about 3.8° to about 7.8°. In some embodiments, θ₃ is about 7.8°. In some embodiments, the forward segment 320 has a length along the first axis A that is from about 1 mm to about 5 mm. For example, in some embodiments the length of forward segment 320 is from about 1.7 mm to about 3.5 mm.

Referring to FIG. 3B, in some embodiments of implants of use in the present method, the second member 310 includes an upright segment 335 that extends from the third member or heel 330 generally along the direction of the second axis B. In various embodiments, the second member 310 further includes a head segment 340 that attaches to the upright segment 335 at an end opposite to the third member or heel 330. In some embodiments, the second member 310 is configured such that the upright segment 335 resides in the vertical portion of the canaliculus while the head segment 340 contacts the tissue surrounding the exterior of the punctum when the lacrimal implant 300 is positioned in the lacrimal canaliculus. In an exemplary embodiment, illustrated in FIGS. 3A-3F, the upright segment 335 has a cylindrical shape and the head segment 340 has an oval or oblong configuration. However, it will be appreciated that any other suitable shapes or configurations can be used and are within the scope of the present invention. For example, in various embodiments, the upright segment 335 is configured to be a conical; the head segment 340 is configured to have a circular, elliptical or polygonal cross section.

In some embodiments, the upright segment 335 has a characteristic diameter that is from about 0.7 mm to about 0.9 mm. For example, in some embodiments, the characteristic diameter of the upright segment 335 is about 0.8 mm.

In some embodiments, the upright segment 335 has a length in the direction of the second axis B that is from about 0.7 mm to about 1.5 mm. For example, in some embodiments the length of upright segment 335 along the direction of the second axis B is about 0.9 mm.

Generally, the head segment 340 has a cross section characterized by a minor axis and a major axis. The minor axis and the major axis refer to the shortest characteristic diameter and the longest characteristic diameter of the cross section, respectively. As such, the minor axis is equal to or less than the major axis. For instance, in some embodiments where the head segment 340 has a circular cross section, the minor axis and the major axis are of equal length. In various embodiments, the head segment 340 has an oval or oblong cross section, and the minor axis is shorter than the major axis. In some embodiments, the head segment 340 is elongated in a direction that is parallel to the first axis A. The major axis indicates the extension of the first member 305 and facilitates positioning of the lacrimal implant 300 in the punctum and canaliculus. In some embodiments, the major axis is from about 1.5 mm to about 2.5 mm. In various embodiments, the minor axis is from about 1 mm to about 1.5 mm. For example, in some embodiments, the major axis and the minor axis head segment 340 are approximately 1.9 mm and 1.3 mm respectively. In some embodiments, the head segment 340 has a thickness in the direction of the second axis that is from about 0.2 mm to about 0.4 mm. For example, in some embodiments, the thickness of the head segment 340 in the direction of the second axis is approximately 0.3 mm.

Referring still to FIG. 3B, exemplary head segment 340 comprises an under-surface 350 facing towards the third member or heel 330 and an outer-surface 355 that faces away from the third member or heel 330. Exemplary head segment 340 further comprises an edge surface 345 that couples the under-surface 350 and the outer-surface 355. The distance between the under-surface 350 and the outer-surface 355 can be readily varied. In some embodiments, the distance is from about 0.2 mm to about 0.4 mm.

In some embodiments, the outer-surface 355 is smaller than the under-surface 350 and is substantially flat. In various embodiments, the edge surface 345 is tapered, curved, angular, or multifaceted. In some embodiments, the edge surface 345 has a radius of curvature that is from about 0.2 mm to about 0.7 mm. In some embodiments, the under-surface 350 is in general flat and is configured to contact the exterior tissue surrounding the punctum when the lacrimal implant 300 is positioned in the lacrimal canaliculus.

In some embodiments, the third member or heel 330 includes an upper surface 360 a lower surface 365 and side surfaces 370. In the illustrated embodiments, the bore 385 extends from the upper surface 360 into the third member or heel 330. In some embodiments, the upper surface 360 and the lower surface 365 are substantially flat and separated from each other by a distance. Such distance is readily variable and is typically about 0.3 mm to about 0.7 mm. For instance, in some embodiments, the upper surface 360 and the lower surface 365 are separated by a distance that is from about 0.4 mm to 0.6 mm (e.g., about 0.53 mm). In some embodiments, the upper surface 360 extends beyond the intersection with the second member 310. In some embodiments, the upper surface 360 extends beyond the intersection with the second member 310 for a distance that is from about 0.3 to about 0.6 mm. The upper surface 360 can also be joined with the side surfaces 370. In various embodiments, upper surface 360 and side surfaces 370 are joined by a curved intersection 380. In some embodiments, the curved intersection 380 has a radius of curvature that is from about 0.04 mm to about 0.08 mm.

Referring now to FIGS. 3D and 3F, in some embodiments, the third member or heel 330 includes a heel connecting segment 375 configured to couple the third member or heel 330 to the first member 305 or to the intermediate segment 315 of the first member 305. The heel connecting segment 375 is of readily variable shape, including flat or curved structures. In FIG. 3F, a width of the heel connecting segment 375 in the direction of the second axis B varies along the direction of the first axis A. For example, the heel connecting segment 375 has a smaller width at or near the side surfaces 370 than the diameter of the intermediate segment 315 of the first member 305. In some embodiments, at or near the intersection with the intermediate segment 315, the heel connecting segment 375 increases the width and thus forms a notch as depicted in FIG. 3F. It will be appreciated that the notch can be either deeper or shallower along both the first axis A and the second axis B before it meets the first member 305 or the second member 310.

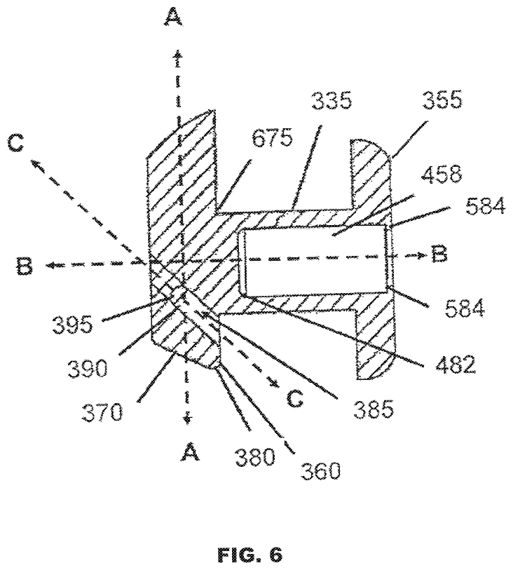

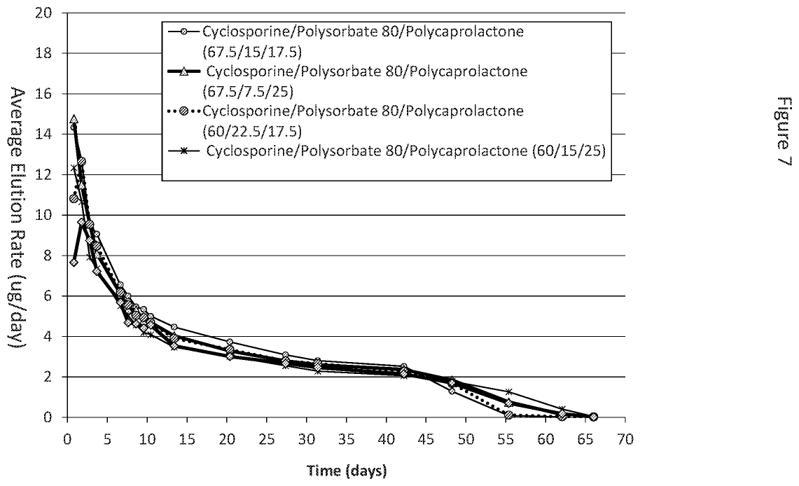

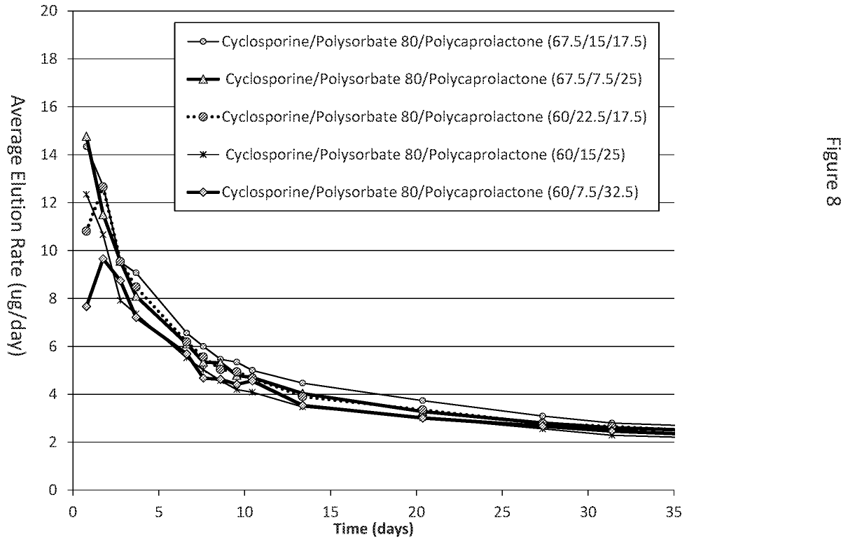

A notch is not a required feature in the implants of the present invention. In some embodiments, the heel connecting segment 375 has the same dimension as the diameter of the intermediate segment 315. For example, the thickness of the third member or heel 330 along the second axis B is equal to the diameter of the intermediate segment 315 of the first member 305. For example, in some embodiments, both the thickness of the third member or heel 330 in the direction of the second axis B and the diameter of the intermediate segment 315 are from about 0.53 mm to about 0.63 mm. In such configurations, the third member or heel 330 couples with the intermediate segment 315 without forming a notch, as illustrated by the alternative heel connecting segment 675 in FIG. 6.

By way of illustration, the third member or heel 330 depicted in FIGS. 3A-3F is substantially parallel to the first axis A of the first member 305. It would be appreciated that this is unnecessary. In some embodiments, the third member or heel 330 can form an angle with relation to the first axis A.

Exemplary structures of the bore 385 are detailed in FIGS. 3E and 3F, where a cross sectional view and a partial enlarged cross-sectional view of the lacrimal implant 300 are provided. The bore 385 is configured to receive a tip or other protrusion of an external insertion tool for facilitating insertion of the lacrimal implant 300 into a lacrimal punctum. The configuration, including size, shape, angle (θ₂) and position of the bore in the heel are readily adjustable to facilitate the mating of the insertion tool with the bore, the flexibility of the heel, or the retention of the lacrimal implants. Depending on the purpose or use of the implant and the materials used for making the heel, the characteristics of the bore noted above are readily varied. Configurations of the bore 385 disclosed herein are illustrative and any other suitable configurations are within the scope of the present invention.

In FIG. 3F, an exemplary bore 385 is characterized by a third axis C and a second angle θ₂ that is defined by the first axis with respect to the third axis A in a similar way as the first angle θ₁. In some embodiments, the second angle θ₂ is from about 15° to about 90°. For example, in some embodiments, the second angle θ₂ is about 45°.

In some embodiments, the bore 385 has a depth along the direction of the third axis C that is from about 0.3 mm to about 0.7 mm. For example, in some embodiments the depth of the bore 385 is approximately 0.4 mm and in some embodiments is approximately 0.6 mm. The bore 385 may include a bore shaft 390 that is generally cylindrical, with a circular, elliptical, oval, or polygonal cross section. The bore 385 may further include a bore tip 395 at which the bore shaft 390 terminates. An exemplary bore tip 395 generally has a semispherical configuration. In some embodiments, the bore shaft 390 has a characteristic diameter that is from about 0.1 mm to about 0.3 mm. In some embodiments, the characteristic diameter of the bore is approximately 0.17 mm. As will be appreciated, the shapes, sizes, orientations disclosed in the present application are illustrative, and any other suitable shapes, sizes, or orientations are within the scope of the present application. In addition, it will be appreciated that the opening of the bore can be positioned closer to the second member or closer to the edge of the heel.

FIG. 4A-4C illustrates an exemplary lacrimal implant 400 that is insertable through a lacrimal punctum 212, 214 and into its associated canaliculus 208, 210. In FIG. 4A, the lacrimal implant 400 comprises a cavity 458 that is configured to house a therapeutic agent core or other materials for release into an eye or surrounding tissues for treatment of various ocular, sinus or other diseases.

In the illustrated exemplary embodiment, the cavity 458 is formed in the head segment 340 and has an opening through the outer-surface 355. The cavity 458 can be shallow such that it stays within the head segment 340. The cavity 458 can be also deeper and extend beyond the head segment 340 and into the upright segment 335. Illustrated exemplary cavity 458 is in general substantially cylindrical with a circular cross section. Any other suitable configuration is within the scope of the present application. For example, in some embodiments, the cavity 458 has a truncated spherical configuration, or has a cylindrical configuration with an oblong or a polygonal cross section.

In some embodiments, the cavity 458 has a depth in the direction of the second axis B that is about from 0.2 mm to about 1.4 mm. For example, in some embodiments, the depth of the cavity 458 is approximately 1.2 mm. In some embodiments, the cavity 458 has a diameter that is from about 0.3 mm to about 0.7 mm. For example, in some embodiments the diameter of the cavity 458 is from about 0.42 mm to about 0.55 mm. In an exemplary embodiment, the cavity 458 extends into the upright segment 335, and the diameter of the cavity 458 is smaller than the diameter of the upright segment 335.

Referring to FIG. 4C, the cavity 458 includes a bottom 482. In various embodiments, the bottom 482 is rounded. In various embodiments, the rounded bottom has a radius of curvature that is from about 0.03 mm to about 0.07 mm.

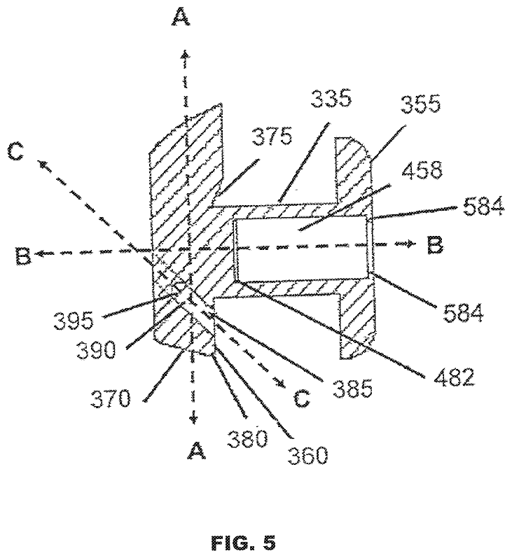

FIG. 5 depicts exemplary configurations of the cavity 458. In FIG. 5, the cavity 458 includes a lip 584 or other retaining structure positioned at the opening of the cavity 458. The lip 584 or the other retaining structure are optionally configured to partially enclose the cavity 458, e.g, prevent a therapeutic agent core or other materials from moving out of the cavity 458. In some embodiments, the lip 584 is a square cross-sectional annulus that extends down from the outer-surface 355 into the cavity 458 and extends inwardly towards the center of the opening of the cavity 458. In some embodiments, the lip 584 is of a tab configuration and includes a plurality of spaced lips that extend inwardly into the opening of the cavity 458. The lip 584 may extend downwardly from about 0.02 mm to about 0.1 mm and inwardly from about 0.02 mm to about 0.1 mm. For example, in some embodiments, the lip 584 extends about 0.05 mm downwardly or inwardly.

Exemplary lacrimal implants of use in methods of the present invention are made of various materials including plastic, rubber, polymer, or composite. Exemplary lacrimal implants of the present invention formed from one or more material including plastic, rubber, polymer, composites, or other appropriate materials. In some embodiments, the lacrimal implants are formed from liquid silicone rubber. For instance, in exemplary embodiments, lacrimal implants are formed from a material marketed as NuSil 4840 liquid silicone rubber, NuSil 4870, or a mixture including such a liquid silicone rubber. Examples of such a mixture include a material marketed as 6-4800, which comprises NuSil 4840 with from about 1% to about 5%, e.g., from about 2% to about 4% 6-4800.

In some embodiments, the lacrimal implant is formed from biodegradable materials, for instance, biodegradable elastic materials including cross-linked polymers, such as poly (vinyl alcohol). In some embodiments, the lacrimal implant can comprise a co-polymer, such as silicone/polyurethane co-polymer, silicone/urethane, silicone/poly (ethylene glycol) (PEG), and silicone/2hydroxyethyl methacrylate (HEMA). As discussed in commonly-owned Utkhede et al., U.S. patent application Ser. No. 12/231,986, entitled “DRUG CORES FOR SUSTAINED RELEASE OF THERAPEUTIC AGENTS,” filed Sep. 5, 2008, which is herein incorporated by reference in its entirety, urethane-based polymer and copolymer materials allow for a variety of processing methods and bond well to one another.

The hardness of the material is selected to facilitate or alter the retention of the lacrimal implant within the lacrimal punctum and its associated canaliculus. Accordingly, in some embodiments, a material having a durometer rating of from about 20 D to about 80 D, e.g., about 30 D to about 70 D, e.g., from about 40 D to about 60 D is of use to adjust parameters such as patient comfort and retention. For example, in some embodiments, the durometer rating of the material used to form the lacrimal implants is approximately 40 D. Materials other than those exemplified above providing a durometer rating for the lacrimal implants within the stated ranges, and particularly that is about 4 0D are also of use. In some embodiments, a harder material or softer material is utilized for the entire lacrimal implant or for portions thereof. In such case, the lacrimal implants are formed from the materials that provide a durometer rating of about 70 D.

In some embodiments, the lacrimal implants of use in the present methods are formed of multiple materials, where certain members or portions of the lacrimal implants are formed with materials having different properties. For example, in some embodiments the first member 305 is formed of a harder durometer rated material while the second member 310 is formed of a softer durometer rated material. In some embodiments, the first member 305 is formed of a softer durometer rated material while the second member 310 is formed of a harder durometer rated material. In some embodiments the third member or heel 330 is formed of a harder durometer rated material than one or more parts of the remainder of the second member 310. In various embodiments, the third member or heel 330 is formed of a softer durometer rated material than the remainder of the second member 310.

Exemplary implants of use in the invention can be formed by methods known in the art, including, but not limited to, machining a blank to the desired shape and size and molding the material forming the implant.

The implant can be one of any number of different designs that releases anti-inflammatory agents and or drugs for a sustained period of time. The disclosures of the following patent documents, which disclose example implant structure or processing embodiments for use in the methods of embodiments of the current invention and methods of making those implants, are incorporated herein by reference in their entirety: U.S. Application Ser. No. 60/871,864 (filed Dec. 26, 2006 and entitled Nasolacrimal Drainage System Implants for Drug Therapy); U.S. application Ser. No. 11/695,537 (filed Apr. 2, 2007 and entitled Drug Delivery Methods, Structures, and Compositions for Nasolacrimal System); U.S. application Ser. No. 12/332,219 (filed Dec. 10, 2008 and entitled Drug Delivery Methods, Structures, and Compositions for Nasolacrimal System); U.S. Application Ser. No. 60/787,775 (filed Mar. 31, 2006 and entitled Nasolacrimal Drainage System Implants for Drug Therapy); U.S. application Ser. No. 11/695,545 (filed Apr. 2, 2007 and entitled Nasolacrimal Drainage System Implants for Drug Therapy); U.S. Application Ser. No. 60/585,287 (filed Jul. 2, 2004 and entitled Treatment Medium Delivery Device and Methods for Delivery of Such Treatment Mediums to the Eye Using Such a Delivery Device); U.S. application Ser. No. 11/571,147 (filed Dec. 21, 2006 and entitled Treatment Medium Delivery Device and Methods for Delivery of Such Treatment Mediums to the Eye Using Such a Delivery Device); U.S. Application Ser. No. 60/970,696 (filed Sep. 7, 2007 and entitled Expandable Nasolacrimal Drainage System Implants); U.S. Application Ser. No. 60/974,367 (filed Sep. 21, 2007 and entitled Expandable Nasolacrimal Drainage System Implants); U.S. Application Ser. No. 60/970,699 (filed Sep. 7, 2007 and entitled Manufacture of Drug Cores for Sustained Release of Therapeutic Agents); U.S. Application Ser. No. 60/970,709 (filed Sep. 7, 2007 and entitled Nasolacrimal Drainage System Implants for Drug Delivery); U.S. Application Ser. No. 60/970,720 (filed Sep. 7, 2007 and entitled Manufacture of Expandable Nasolacrimal Drainage System Implants); U.S. Application Ser. No. 60/970,755 (filed Sep. 7, 2007 and entitled Prostaglandin Analogues for Implant Devices and Methods); U.S. Application Ser. No. 60/970,820 (filed Sep. 7, 2007 and entitled Multiple Drug Delivery Systems and Combinations of Drugs with Punctal Implants); U.S. Application Ser. No. 61/066,223 (filed Feb. 18, 2008 and entitled Lacrimal Implants and Related Methods); U.S. Application Ser. No. 61/049,347 (filed Apr. 30, 2008 and entitled Lacrimal Implants and Related Methods); U.S. Application Ser. No. 61/033,211 (filed Mar. 3, 2008 and entitled Lacrimal Implants and Related Methods); U.S. Application Ser. No. 61/049,360 (filed Apr. 30, 2008 and entitled Lacrimal Implants and Related Methods); U.S. Application Ser. No. 61/052,595 (filed May 12, 2008 and entitled Lacrimal Implants and Related Methods); U.S. Application Ser. No. 61/075,309 (filed Jun. 24, 2008 and entitled Lacrimal Implants and Related Methods); U.S. Application Ser. No. 61/154,693 (filed Feb. 23, 2009 and entitled Lacrimal Implants and Related Methods); U.S. Application Ser. No. 61/209,036 (filed Mar. 2, 2009 and entitled Lacrimal Implants and Related Methods); U.S. Application Ser. No. 61/209,630 (filed Mar. 9, 2009 and entitled Lacrimal Implants and Related Methods); U.S. Application Ser. No. 61/036,816 (filed Mar. 14, 2008 and entitled Lacrimal Implants and Related Methods); U.S. Application Ser. No. 61/271,862 (filed Jul. 27, 2009 and entitled Lacrimal Implants and Related Methods); U.S. Application Ser. No. 61/252,057 (filed Oct. 15, 2009 and entitled Lacrimal Implants and Related Methods); U.S. application Ser. No. 12/710,855 (filed Feb. 23, 2010 and entitled Lacrimal Implants and Related Methods); U.S. Application Ser. No. 60/871,867 (filed Dec. 26, 2006 and entitled Drug Delivery Implants for Inhibition of Optical Defects); U.S. application Ser. No. 12/521,543 (filed Dec. 31, 2009 and entitled Drug Delivery Implants for Inhibition of Optical Defects); U.S. Application Ser. No. 61/052,068 (filed May 9, 2008 and entitled Sustained Release Delivery of Latanoprost to Treat Glaucoma); U.S. Application Ser. No. 61/052,113 (filed May 9, 2008 and entitled Sustained Release Delivery of Latanoprost to Treat Glaucoma); U.S. Application Ser. No. 61/108,777 (filed Oct. 27, 2008 and entitled Sustained Release Delivery of Latanoprost to Treat Glaucoma); U.S. application Ser. No. 12/463,279 (filed May 8, 2009 and entitled Sustained Release Delivery of Active Agents to Treat Glaucoma and Ocular Hypertension); U.S. Application Ser. No. 61/049,337 (filed Apr. 30, 2008 and entitled Lacrimal Implants and Related Methods); U.S. application Ser. No. 12/432,553 (filed Apr. 29, 2009 and entitled Composite Lacrimal Insert and Related Methods); U.S. Application Ser. No. 61/049,317 (filed Apr. 30, 2008 and entitled Drug-Releasing Polyurethane Lacrimal Insert); U.S. application Ser. No. 12/378,710 (filed Feb. 17, 2009 and entitled Lacrimal Implants and Related Methods); U.S. Application Ser. No. 61/075,284 (filed Jun. 24, 2008 and entitled Combination Treatment of Glaucoma); U.S. application Ser. No. 12/490,923 (filed Jun. 24, 2009 and entitled Combination Treatment of Glaucoma); U.S. Application Ser. No. 61/134,271 (filed Jul. 8, 2008 and entitled Lacrimal Implant Body Including Comforting Agent); U.S. application Ser. No. 12/499,605 (filed Jul. 8, 2009 and entitled Lacrimal Implant Body Including Comforting Agent); U.S. Application Ser. No. 61/057,246 (filed May 30, 2008 and entitled Surface Treatment of Implants and Related Methods); U.S. Application Ser. No. 61/132,927 (filed Jun. 24, 2008 and entitled Surface Treated Implantable Articles and Related Methods); U.S. application Ser. No. 12/283,002 (filed Sep. 5, 2008 and entitled Surface Treated Implantable Articles and Related Methods); U.S. application Ser. No. 12/231,989 (filed Sep. 5, 2008 and entitled Lacrimal Implants and Related Methods); U.S. Application Ser. No. 61/049,317 (filed Apr. 30, 2008 and entitled Drug-Releasing Polyurethane Lacrimal Insert); U.S. application Ser. No. 12/231,986 (filed Sep. 5, 2008 and entitled Drug Cores for Sustained Release of Therapeutic Agents); U.S. Application Ser. No. 61/050,901 (filed May 6, 2008 and entitled Punctum Plug Detection); U.S. application Ser. No. 12/231,987 (filed Sep. 5, 2008 and entitled Lacrimal Implant Detection); U.S. Application Ser. No. 61/146,860 (filed Jan. 23, 2009 and entitled Sustained Release Delivery of One or More Anti-Glaucoma Agents); U.S. Application Ser. No. 61/152,909 (filed Feb. 16, 2009 and entitled Sustained Release Delivery of One or More Anti-Glaucoma Agents); U.S. Application Ser. No. 61/228,894 (filed Jul. 27, 2009 and entitled Sustained Release Delivery of One or More Anti-Glaucoma Agents); U.S. Application Ser. No. 61/277,000 (filed Sep. 18, 2009 and entitled Drug Cores for Sustained Ocular Release of Therapeutic Agents); U.S. application Ser. No. 12/692,452 (filed Jan. 22, 2010 and entitled Sustained Release Delivery of One or More Agents); U.S. Application Ser. No. 61/283,100 (filed Nov. 27, 2009 and entitled Lacrimal Implants Including Split and Insertable Drug Core); International Application Serial No. PCT/US2010/058129 (filed Nov. 26, 2010, published as WO 2011/066479 and entitled Lacrimal Implants Including Split and Insertable Drug Core); U.S. Application Ser. No. 61/139,456 (filed Dec. 19, 2008 and entitled Substance Delivering Punctum Implants and Methods); U.S. application Ser. No. 12/643,502 (filed Dec. 21, 2009 and entitled Substance Delivering Punctum Implants and Methods); U.S. application Ser. No. 10/825,047 (filed Apr. 15, 2004 and entitled Drug Delivery via Punctal Plug); U.S. application Ser. No. 12/604,202 (filed Oct. 22, 2009 and entitled Drug Delivery via Ocular Implant); International Application Serial No. PCT/US2005/023848 (filed Jul. 1, 2005, published as WO 2006/014434 and entitled Treatment Medium Delivery Device and Methods for Delivery); International Application Serial No. PCT/US2007/065792 (filed Apr. 2, 2007, published as WO 2007/115261 and entitled Drug Delivery Methods, Structures, and Compositions for Nasolacrimal System); and International Application Serial No. PCT/US2007/065789 (filed Apr. 2, 2007, published as WO 2007/115259 and entitled Nasolacrimal Drainage System Implants for Drug Therapy).

In various embodiments of the methods of the invention, an implant including a retention structure is employed to retain the implant in the punctum or canaliculus. The retention structure is attached to or integral with the implant body. The retention structure comprises an appropriate material that is sized and shaped so that the implant can be easily positioned in the desired tissue location, for example, the punctum or canaliculus. In some embodiments, the drug core may be attached to the retention structure via, at least in part, the sheath. In some embodiments, the retention structure comprises a hydrogel configured to expand when the retention structure is placed in the punctum. The retention structure can comprise an attachment member having an axially oriented surface. In some embodiments, expansion of the hydrogel can urge against the axially oriented surface to retain the hydrogel while the hydrogel is hydrated. In some embodiments, the attachment member can comprise at least one of a protrusion, a flange, a rim, or an opening through a portion of the retention structure. In some embodiments, the retention structure includes an implant body portion size and shape to substantially match an anatomy of the punctum and canaliculus.

The retention structure may have a size suitable to fit at least partially within the canalicular lumen. The retention structure can be expandable between a small profile configuration suitable for insertion and a large profile configuration to anchor the retention structure in the lumen, and the retention structure can be attached near the distal end of the drug core. In specific embodiments, the retention structure can slide along the drug core near the proximal end when the retention structure expands from the small profile configuration to the large profile configuration. A length of the retention structure along the drug core can be shorter in the large profile configuration than the small profile configuration.

In some embodiments, the retention structure is resiliently expandable. The small profile may have a cross section of no more than about 0.2 mm, and the large profile may have a cross section of no more than about 2.0 mm. The retention structure may comprise a tubular body having arms separated by slots. The retention structure can be disposed at least partially over the drug core.

In some embodiments, the retention structure is mechanically deployable and typically expands to a desired cross-sectional shape, for example with the retention structure comprising a super elastic shape memory alloy such as Nitinol™. Other materials in addition to Nitinol™ can be used, for example resilient metals or polymers, plastically deformable metals or polymers, shape memory polymers, and the like, to provide the desired expansion. In some embodiments polymers and coated fibers available from Biogeneral, Inc. of San Diego, Calif. may be used. Many metals such as stainless steels and non-shape memory alloys can be used and provide the desired expansion. This expansion capability permits the implant to fit in hollow tissue structures of varying sizes, for example canaliculae ranging from 0.3 mm to 1.2 mm (i.e. one size fits all). Although a single retention structure can be made to fit canaliculae from 0.3 to 1.2 mm across, a plurality of alternatively selectable retention structures can be used to fit this range if desired, for example a first retention structure for canaliculae from 0.3 to about 0.9 mm and a second retention structure for canaliculae from about 0.9 to 1.2 mm. The retention structure has a length appropriate to the anatomical structure to which the retention structure attaches, for example a length of about 3 mm for a retention structure positioned near the punctum of the canaliculus. For different anatomical structures, the length can be appropriate to provide adequate retention force, e.g. 1 mm to 15 mm lengths as appropriate.

Although the implant body may be attached to one end of the retention structure as described above, in many embodiments the other end of the retention structure is not attached to the implant body so that the retention structure can slide over the implant body including the sheath body and drug core while the retention structure expands. This sliding capability on one end is desirable as the retention structure may shrink in length as the retention structure expands in width to assume the desired cross-sectional width. However, it should be noted that many embodiments may employ a sheath body that does not slide in relative to the core.

In many embodiments, the retention structure can be retrieved from tissue. A projection, for example a hook, a loop, or a ring, can extend from a portion of the implant body to facilitate removal of the retention structure.

In some embodiments the sheath and retention structure can comprise two parts.

The lacrimal implants of the present invention have exceptional retention properties and are retained in the punctum and canaliculus for a period that is enhanced relative to a commercially available plug based upon the percentage of eyes in which an implant was implanted retaining the implant over a selected time period.

In an exemplary embodiment, the method of the invention uses a lacrimal implant configured to remain implanted in a punctum for at least about 1 week, 2 weeks, 3 weeks, 4 weeks, 5 weeks, 6 weeks, 7 weeks, 8, weeks, 9 weeks 10 weeks, 11 weeks, or at least about 12 weeks or more. In an exemplary embodiment, the lacrimal implant is configured to be retained by the puncta for the duration of the intended sustained release of the therapeutic agent. In various embodiments, the duration of the intended sustained release of the therapeutic agent is at least about 1 week, 2 weeks, 3 weeks, 4 weeks, 5 weeks, 6 weeks, 7 weeks, 8, weeks, 9 weeks 10 weeks, 11 weeks, or at least about 12 weeks or more. In various embodiments at least about 95%, at least about 90%, at least about 85% or at least about 80% of the implanted implants are retained for the duration of the intended controlled release of the therapeutic agent. In an exemplary embodiment, the implant is retained by the puncta for a length of time to show therapeutic efficacy.

In various embodiments, the present invention provides for the use of implants having structural features that enhance the retention of the implant in a punctum. Amongst other features, the heel of the present implant (e.g., 330) is configured to come to rest in the lacrimal canaliculus ampulla (e.g., 252), effectively locking the implant into place. However, the inventors have recognized that to prevent rotation and relative movement of the implanted device, which plays a role in the displacement of the device, a first member was needed to maintain the heel in the ampulla. Thus, the first member, e.g., 305, is configured to stabilize the punctal plug within the lacrimal canaliculus, prevent rotation and maintain positioning of the plug when the surrounding tissue moves.

In an exemplary embodiment, the methods of the invention use an implant having an occlusive element. An occlusive element can be mounted to and expandable with the retention structure to inhibit tear flow. An occlusive element may inhibit tear flow through the lumen, and the occlusive element may cover at least a portion of the retention structure to protect the lumen from the retention structure. The occlusive element comprises an appropriate material that is sized and shaped so that the implant can at least partially inhibit, even block, the flow of fluid through the hollow tissue structure, for example lacrimal fluid through the canaliculus. The occlusive material may be a thin walled membrane of a biocompatible material, for example silicone, that can expand and contract with the retention structure. The occlusive element is formed as a separate thin tube of material that is slid over the end of the retention structure and anchored to one end of the retention structure as described above. Alternatively, the occlusive element can be formed by dip coating the retention structure in a biocompatible polymer, for example silicone polymer. The thickness of the occlusive element can be in a range from about 0.01 mm to about 0.15 mm, and often from about 0.05 mm to 0.1 mm.

### Methods of Use

In embodiments, provided herein are methods for delivering an ophthalmic drug to an eye for dry-eye treatment, comprising: placing a medical device disclosed herein comprising any one of the solid matrix sustained release ophthalmic formulations disclosed herein, on, in or near the eye of a patient, wherein the ophthalmic drug is cyclosporine. In certain embodiments, the medical device is a lacrimal implant, wherein the lacrimal implant is placed through a punctum and into a canalicular lumen of a patient. In certain other embodiments, the medical device is an intracanalicular plug, wherein the intracanalicular plug is placed through a punctum and into a canalicular lumen of a patient. In other embodiments, the medical device is an ocular ring, wherein the ring is placed on the surface of the eye and under the eye lid (outside the field of vision).

In embodiments, treatment period for dry eye is about one month to about 6 months.

The methods of the present invention can be administered to a mammal in need of treatment by way of a variety of routes. For example, drug delivery systems may be used by implantation within a portion of the body in need of localized drug delivery, e.g., the interior portion of an eye. However, the exemplary matrix-controlled diffusion drug delivery systems may likewise be used in accordance with other surgical procedures known to those skilled in the field of ophthalmology. For example, the drug delivery systems can be administered to the region of the eye in need of treatment employing instruments known in the art, e.g., a flexible microcatheter system or cannula disclosed in U.S. Patent Application Publication No. 2002/0002362, or the intraretinal delivery and withdrawal systems disclosed in U.S. Pat. Nos. 5,273,530 and 5,409,457, the contents of each which are incorporated by reference herein. The pharmaceutically active agent may be released from the drug delivery device over a sustained and extended period of time. Optionally, the drug release rate may also be controlled through the attachment of an inert diffusion barrier by way of, for example, surface treatment of the drug delivery device. The surface treatment may be applied through a variety of surface treatment techniques known in the art, e.g., oxidative plasma, evaporative deposition, dip coating or extrusion techniques.

### Optional Formulation Components

The present formulation may further comprise a pharmaceutically acceptable carrier, e.g., excipients, suspending agents, diluents, fillers, salts, buffers, stabilizers, solubilizers, solvents, dispersion media, coatings, isotonic agents, and other materials known in the art. The pharmaceutical formulation optionally includes potentiators, complexing agents, targeting agents, stabilizing agents, cosolvents, pressurized gases, or solubilizing conjugates.

Exemplary excipients include sugars such as lactose, sucrose, mannitol, or sorbitol; cellulose preparations such as, maize starch, wheat starch, rice starch, potato starch, gelatin, gum tragacanth, methyl cellulose, hydroxypropylmethylcellulose, sodium caroxymethylcellulose, and/or polyvinylpyrrolidone (PVP). Preferred excipients include lactose, gelatin, sodium carboxymethyl cellulose, and low molecular weight starch products.

Exemplary suspending agents that can serve as valve lubricants in pressurized pack inhaler systems are desirable. Such agents include oleic acid, simple carboxylic acid derivatives, and sorbitan trioleate.

Exemplary diluents include water, saline, phosphate-buffered citrate or saline solution, and mucolytic preparations. Other diluents that can be considered include alcohol, propylene glycol, and ethanol; these solvents or diluents are more common in oral aerosol formulations. Physiologically acceptable diluents that have a tonicity and pH compatible with the alveolar apparatus are desirable. Preferred diluents include isotonic saline, phosphate buffered isotonic solutions whose tonicity have been adjusted with sodium chloride or sucrose or dextrose or mannitol.

Exemplary fillers include glycerin, propylene glycol, ethanol in liquid or fluid preparations. Suitable fillers for dry powder inhalation systems include lactose, sucrose, dextrose, suitable amino acids, and derivatives of lactose. Preferred fillers include glycerin, propylene glycol, lactose and certain amino acids.

Exemplary salts include those that are physiologically compatible and provide the desired tonicity adjustment. Monovalent and divalent salts of strong or weak acids are desirable. Preferred salts include sodium chloride, sodium citrate, ascorbates, sodium phosphates.

Exemplary buffers include phosphate or citrate buffers or mixed buffer systems of low buffering capacity. Preferred buffers include phosphate or citrate buffers.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by weight, and temperature is in degrees Centigrade. It should be understood that variations in the methods as described can be made without changing the fundamental aspects that the Examples are meant to illustrate.

### Example 1: Manufacturing of Cyclosporine Sustained Release Formulation in a Hydrophobic Polymer and Nonionic Surfactant Solid Matrix

Cyclosporine, 1.0-1.3 dL/g intrinsic viscosity poly(caprolactone) (PCL) and polysorbate 80 (PS80) at the target weight ratio were dissolved via mixing at 50-60° C. in a sufficient volume of tetrahydrofuran in a suitably sized round bottom flask. In this example, Cyclosporine was present from about 60 to 80% (w/w); polycaprolactone from about 14 to 32% (w/w); and polysorbate 80 from about 4.5 to 22.5% (w/w). After dissolution, the round bottom flask (RBF) was installed on a rotary evaporator to remove the bulk THF. Residual THF was removed by storage in vacuum oven at 70-75° C., for a minimum of 19.5 hours at −27inHg.

The mixture was injected into polyimide tubing (ID of 0.022 inches, OD of 0.024 inches) at a temperature of approximately 85° C. to liquefy the mixture. The polyimide tubing filled with the Cyclosporine mixture was cooled to room temperature (e.g. 20 to 25° C.). The filled tubing was cut into 0.95 mm section lengths to form the drug core and the distal end was sealed with a small volume of UV cured methacrylate adhesive.

The glued drug cores were inserted glue dome first into the lumen of silicone punctal plugs in preparation for an elution rate testing procedure. For elution testing, the loaded punctal plugs were placed into individual 2 mL glass vials with screw-on caps. About 1.0 mL of elution buffer comprised of 0.1% by weight sodium dodecyl sulfate detergent dissolved in phosphate-buffered saline. The vial was then capped and mixed at 100 rpm in a shaker-incubator at 30-35° C. After one day of mixing, the vial was removed, the plug transferred to a fresh vial of elution buffer for the day 2 incubation. The cyclosporine-containing day 1 elution buffer was retained for analysis for cyclosporine content. Serial transfers and incubation of the plug in elution buffer were performed on at least the following target days: 1, 2, 3, 4, 7, 8, 9, 10, 11, 14, 21, 28, 35, 42, 49, 56 and 63. Recorded times and dates for the shaking start times and removal times were used to calculate exact mixing times. The spent plugs at the end of elution testing were retained for analytical determination of their residual cyclosporine contents.

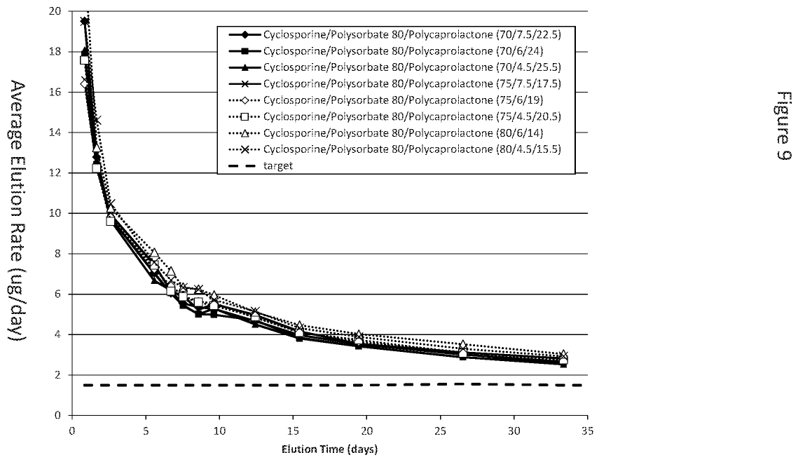

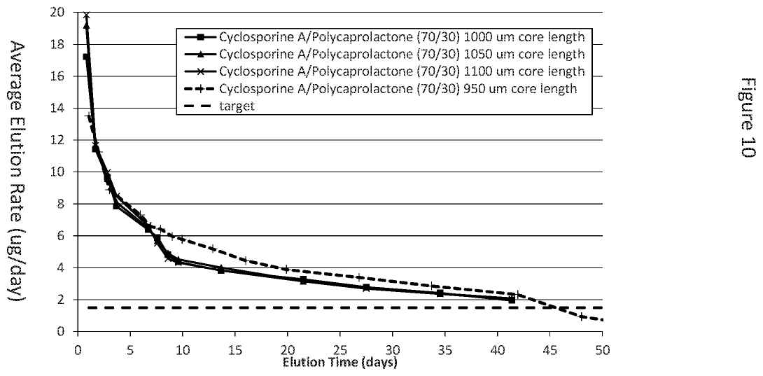

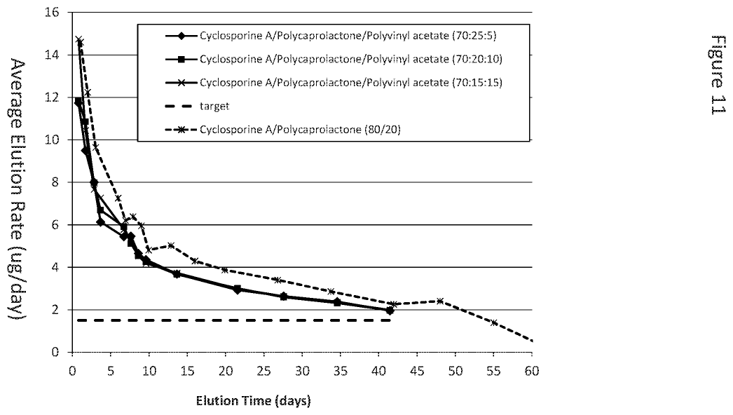

The daily elution rate in μg/mL per day were calculated using the analytically determined cyclosporine concentrations and mixing times for each vial sample. Average daily elution rates were calculated from multiple vials derived from insert samples taken from each extrusion, or section thereof. See FIGS. 7 to 9, wherein the elution was measured from drug cores comprising different ratios of cyclosporine/PS80/PCL. The target elution rate per day, as shown in FIG. 9, is 1.5 μg/day.

### Example 2: Manufacturing of Cyclosporine Sustained Release Formulation in One or More Hydrophobic Polymer Solid Matrix

Cyclosporine, 1.0-1.3 dL/g intrinsic viscosity poly(caprolactone) (PCL) and polyvinyl acetate (PVAc) at the target weight ratio were dissolved via mixing at 50-60° C. in a sufficient volume of tetrahydrofuran in a 20mL vial. In this example, Cyclosporine was present from 70% to 80% (w/w); polycaprolactone from 15 to 25% (w/w); and PVAc from about 0 to 15% (w/w). After dissolution, the solution was transferred to a suitably sized round bottom flask (RBF) and the RBF was installed on a rotary evaporator to remove the bulk THF. Residual THF was removed by storage in vacuum oven at 70-80° C., for a minimum of 19.5 hours at −27 inHg.

The mixture was injected into polyimide tubing (ID of 0.022 inches, OD of 0.024 inches) at a temperature of between approximately 90° C. to 110 ° C. to liquefy the mixture. The polyimide tubing filled with the Cyclosporine mixture was cooled to room temperature (e.g. 20 to 25° C.). The filled tubing was cut into sections from 0.95 to 1.10 mm lengths at an elevated temperature of between approximately 60° C. to 70 ° C. to form the drug core and the distal end was sealed with a small volume of UV cured methacrylate adhesive.

The glued drug cores were inserted glue dome first into the lumen of silicone punctal plugs in preparation for an elution rate testing procedure. For elution testing, the loaded punctal plugs were placed into individual 2 mL glass vials with screw-on caps. About 1.0mL of elution buffer comprised of 0.1% by weight sodium dodecyl sulfate detergent dissolved in phosphate-buffered saline. The vial was then capped and mixed at 100 rpm in a shaker-incubator at 30-35° C. After one day of mixing, the vial was removed, the plug transferred to a fresh vial of elution buffer for the day 2 incubation. The cyclosporine-containing day 1 elution buffer was retained for analysis for cyclosporine content. Serial transfers and incubation of the plug in elution buffer were performed on at least the following target days: 1, 2, 3, 4, 7, 8, 9, 10, 11, 14, 21, 28, 35, 42, 49, 56 and 63. Recorded times and dates for the shaking start times and removal times were used to calculate exact mixing times. The spent plugs at the end of elution testing were retained for analytical determination of their residual cyclosporine contents.

The daily elution rate in μg/mL per day were calculated using the analytically determined cyclosporine concentrations and mixing times for each vial sample. Average daily elution rates were calculated from multiple vials derived from insert samples taken from each extrusion, or section thereof. See FIGS. 10 and 11, wherein the elution was measured from drug cores comprising different ratios of cyclosporine/PCL/polyvinyl acetate. The target elution rate per day, as shown in FIGS. 10 and 11, is 1.5 μg/day.

### Example 3: Manufacturing of Cyclosporine Sustained Release Formulation in One or More Hydrophobic Polymer Solid Matrix With or Without a Surfactant

Cyclosporine, 1.0-1.3 dL/g intrinsic viscosity poly(caprolactone) (PCL), polyvinyl acetate (PVAc) and polysorbate 80 (PS80) at the target weight ratio were dissolved via mixing at 50-60° C. in a sufficient volume of tetrahydrofuran in a 20 mL vial. In this example, Cyclosporine was present at 70 or 75% (w/w); polycaprolactone from 5 to 20% (w/w); PVAc from about 5 to 20% (w/w); and PS80 from about 0 to 3%. After dissolution, the solution was transferred to a suitably sized round bottom flask (RBF) and the RBF was installed on a rotary evaporator to remove the bulk THF. Residual THF was removed by storage in vacuum oven at 70-80° C., for a minimum of 16.5 hours at −27 inHg.

The mixture was injected into polyimide tubing (ID of 0.022 inches, OD of 0.024 inches) at a temperature of between approximately 90° C. to 110 ° C. to liquefy the mixture. The polyimide tubing filled with the Cyclosporine mixture was cooled to room temperature (e.g. 20 to 25° C.). The filled tubing was cut into sections of 1.10 mm lengths at an elevated temperature of between approximately 60° C. to 70 ° C. to form the drug core and the distal end was sealed with a small volume of UV cured methacrylate adhesive.

The glued drug cores were inserted glue dome first into the lumen of silicone punctal plugs in preparation for an elution rate testing procedure. For elution testing, the loaded punctal plugs were placed into individual 2 mL glass vials with screw-on caps. About 1.0 mL of elution buffer comprised of 0.1% by weight sodium dodecyl sulfate detergent dissolved in phosphate-buffered saline. The vial was then capped and mixed at 100 rpm in a shaker-incubator at 30-35° C. After one day of mixing, the vial was removed, the plug transferred to a fresh vial of elution buffer for the day 2 incubation. The cyclosporine-containing day 1 elution buffer was retained for analysis for cyclosporine content. Serial transfers and incubation of the plug in elution buffer were performed on at least the following target days: 1, 2, 3, 4, 7, 8, 9, 10, 11, 14, 21, 28, 35, 42, 49, 56 and 63. Recorded times and dates for the shaking start times and removal times were used to calculate exact mixing times. The spent plugs at the end of elution testing were retained for analytical determination of their residual cyclosporine contents.

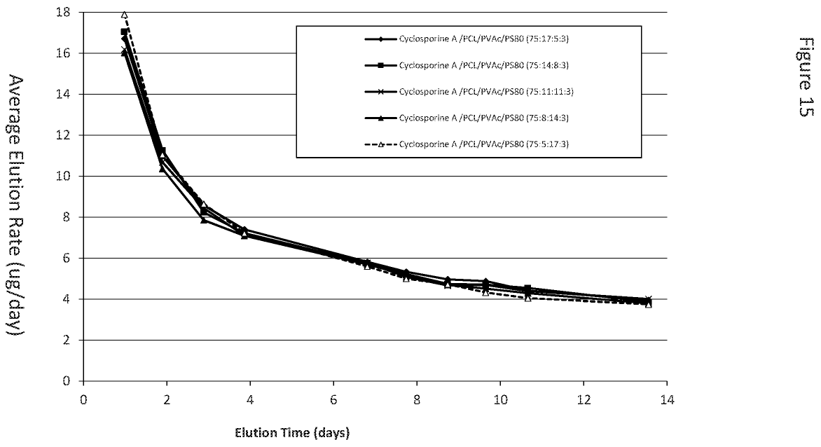

The daily elution rate in μg/mL per day were calculated using the analytically determined cyclosporine concentrations and mixing times for each vial sample. Average daily elution rates were calculated from multiple vials derived from insert samples taken from each extrusion, or section thereof. See FIGS. 12 to 15, wherein the elution was measured from drug cores comprising different ratios of cyclosporine/PCL/polyvinyl acetate. The target elution rate per day, as shown in FIGS. 12 and 13, is 1.5 μg/day.

We claim:

1. A solid matrix sustained release ophthalmic formulation for topical delivery of an ophthalmic drug, comprising:
a) at least one hydrophobic polymer; b) a nonionic surfactant; and, c) the ophthalmic drug, wherein the formulation does not comprise a hydrophilic polymer and the formulation is adapted to release the ophthalmic drug at therapeutically effective levels each day for a period of about two weeks to about 6 weeks.

2. The formulation of claim 1, wherein the solid matrix does not comprise silicone.

3. The formulation of claim 1, wherein the solid matrix does not comprise PEG polymers.

4. The formulation of claim 1, wherein the solid matrix does not comprise a hydrophilic polymer selected from polyethylene glycol (PEG) polymers, acrylate-derivatized PEG (PEGDA) polymers, polysaccharide polymers, hydrophilic polyanhydrides or a combination thereof.

5. The formulation of claim 1, wherein the solid matrix does not comprise methacrylate polymers or monomers.

6. The formulation of claim 1, wherein the hydrophobic polymer comprises silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate) (PVAc), poly(vinyl alcohol) (PVA), poly(vinyl ether), poly(vinyl ketone), poly(vinylpyrrolidone (PVP), poly(vinylpyridine), co-polymers thereof, or combinations thereof.

7. The formulation of claim 1, wherein the hydrophobic polymer is selected from polyester, polycaprolactone, poly(D,L-lactic-co-glycolic acid) (PLGA), poly lactic acid (PLA), poly(vinyl acetate) (PVAc), polyurethane, poly glycolic acid (PGA) or a combination thereof.

8. The formulation of claim 1, wherein the hydrophobic polymer is polycaprolactone.

9. The formulation of claim 7, wherein the polycaprolactone polymer is present from about 12.5 to about 47.5% (w/w).

10. The formulation of claim 7, wherein the polycaprolactone polymer is present from about 14 to about 30% (w/w).

11. The formulation of claim 1, wherein the nonionic surfactant is selected from tyloxapol, a sorbitan ester, polyoxyethylene ethers, a polysorbate or a combination thereof.

12. The formulation of claim 1, wherein the ophthalmic drug is cyclosporine.

13. The formulation of claim 12, wherein the cyclosporine is present from about 20 to about 80% (w/w).

14. The formulation of claim 12, wherein the cyclosporine is present from about 60 to about 80% (w/w).

15. The formulation of claim 1, wherein the solid matrix composition comprises about 60 to about 240 μg of cyclosporine.

16. The formulation of claim 1, wherein the solid matrix composition is configured, when placed within the lacrimal canaliculus, to elute about 1 μg to about 3 μg of cyclosporine a day from about 2 weeks to about 6 weeks.

17. The formulation of claim 1, further comprising a sheath body disposed at least partially over the solid matrix.

18. The formulation of claim 1, wherein the ophthalmic drug is cyclosporine and the solid matrix comprises polycaprolactone, poly(vinyl acetate) (PVAc), and a polysorbate surfactant.

19. The formulation of claim 18, wherein the surfactant is polysorbate 80.

20. The formulation of claim 19, wherein the polysorbate 80 present in the solid matrix from about 0 to about 15% (w/w).

21. The formulation of claim 19, wherein the polysorbate 80 present in the solid matrix from about 0 to about 5% (w/w).

22. The formulation of claim 1, wherein the hydrophobic polymer is polycaprolactone and is present from 15 to 30% (w/w), the nonionic surfactant is polysorbate 80 and is present from 4.5 to 10% (w/w), and the ophthalmic drug is cyclosporine and is present from 70 to 80% (w/w).

23. A sustained release ophthalmic formulation for topical delivery of an ophthalmic drug, comprising:
cyclosporine admixed with a hydrophobic polymer and a nonionic surfactant to form a solid matrix composition, wherein the composition is in the form of a drug core and configured for placement within a lacrimal canaliculus.

24. The formulation of claim 23, adapted to release the cyclosporine at therapeutically effective levels each day for a period of about two weeks to about 6 weeks.

25. The formulation of claim 23, wherein the drug core does not comprise silicone.

26. The formulation of claim 23, wherein the drug core does not comprise PEG polymers.

27. The formulation of claim 23, wherein the drug core does not comprise a hydrophilic polymer selected from polyethylene glycol (PEG) polymers, acrylate-derivatized PEG (PEGDA) polymers, polysaccharide polymers, hydrophilic polyanhydrides or a combination thereof.

28. The formulation of claim 23, wherein the drug core does not comprise methacrylate polymers or monomers.

29. The formulation of claim 23, wherein the hydrophobic polymer comprises silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate)(PVAc), poly(vinyl alcohol) (PVA), poly(vinyl ether), poly(vinyl ketone), poly(vinylpyrrolidone (PVP), poly(vinylpyridine), co-polymers thereof, or combinations thereof.

30. The formulation of claim 23, wherein the hydrophobic polymer is selected from polyester, polycaprolactone, poly(D,L-lactic-co-glycolic acid) (PLGA), poly(vinyl acetate)(PVAc), poly lactic acid (PLA), polyurethane, poly glycolic acid (PGA) or a combination thereof.

31. The formulation of claim 23, wherein the hydrophobic polymer is polycaprolactone.

32. The formulation of claim 31, wherein the polycaprolactone polymer is present from about 12.5 to about 47.5% (w/w).

33. The formulation of claim 31, wherein the polycaprolactone polymer is present from about 14 to about 30% (w/w).

34. The formulation of claim 23, wherein the nonionic surfactant is selected from tyloxapol, a sorbitan ester, polyoxyethylene ethers, a polysorbate or a combination thereof.

35. The formulation of claim 23, wherein the nonionic surfactant is polysorbate 80.

36. The formulation of claim 35, wherein the polysorbate 80 present in the drug core from about 0 to about 25% (w/w).

37. The formulation of claim 35, wherein the polysorbate 80 present in the drug core from about 4.5 to about 10% (w/w).

38. The formulation of claim 23, wherein the cyclosporine is present from about 20 to about 80% (w/w).

39. The formulation of claim 23, wherein the cyclosporine is present from about 60 to about 80% (w/w).

40. The formulation of claim 23, wherein the solid matrix composition comprises about 60 to about 240 μg of cyclosporine.

41. The formulation of claim 23, wherein the drug core composition is configured, when placed within the lacrimal canaliculus, to elute about 1 μg to about 3 μg of the cyclosporine a day from about 2 weeks to about 6 weeks.

42. The formulation of claim 23, further comprising a sheath body disposed at least partially over the drug core.

43. The formulation of claim 23, wherein the drug core comprises polycaprolactone and a polysorbate surfactant.

44. The formulation of claim 1, wherein the hydrophobic polymer is polycaprolactone and is present from 15 to 30% (w/w), the nonionic surfactant is polysorbate 80 and is present from 4.5 to 10% (w/w), and the cyclosporine is present from 70 to 80% (w/w).

45. A lacrimal implant comprising:
a punctal plug comprising a plug body and a drug insert, wherein the insert comprises;
a drug core comprising the formulation according to any one of claim 1-45; and,
an impermeable sheath body partially covering the drug core, wherein the sheath body is configured to provide an exposed proximal end of the drug core in direct contact with tear fluid that releases an ophthalmic drug to the eye when the drug insert is disposed within a channel of the punctal plug and the punctal plug is inserted into the lacrimal canaliculus of a patient.

46. A method for delivering an ophthalmic drug to the eye for treatment of dry eye, comprising:
placing a lacrimal implant through a punctum and into a canalicular lumen of a patient, the implant comprising; a sustained release ophthalmic formulation according to any one of claim 1-45, wherein the ophthalmic drug is cyclosporine.

47. A solid matrix sustained release ophthalmic formulation for topical delivery of an ophthalmic drug, comprising:
a) one or more hydrophobic polymers; and, b) the ophthalmic drug, wherein the formulation does not comprise a hydrophilic polymer or a nonionic surfactant and the formulation is adapted to release the ophthalmic drug at therapeutically effective levels each day for a period of about two weeks to about 8 weeks.

48. The formulation of claim 47, wherein the solid matrix does not comprise silicone.

49. The formulation of claim 47, wherein the solid matrix does not comprise a nonionic surfactant selected from tyloxapol, a sorbitan ester, polyoxyethylene ethers, a polysorbate or a combination thereof.

49. The formulation of claim 47, wherein the solid matrix does not comprise PEG polymers.

50. The formulation of claim 47, wherein the solid matrix does not comprise a hydrophilic polymer selected from polyethylene glycol (PEG) polymers, acrylate-derivatized PEG (PEGDA) polymers, polysaccharide polymers, hydrophilic polyanhydrides or a combination thereof.

51. The formulation of claim 47, wherein the solid matrix does not comprise methacrylate polymers or monomers.

52. The formulation of claim 47, wherein the one or more hydrophobic polymers comprise silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate) (PVAc), poly(vinyl alcohol) (PVA), poly(vinyl ether), poly(vinyl ketone), poly(vinylpyrrolidone (PVP), poly(vinylpyridine), co-polymers thereof, or combinations thereof.

53. The formulation of claim 47, wherein the one or more hydrophobic polymers is selected from polyester, poly(vinyl acetate) (PVAc), polycaprolactone, poly(D,L-lactic-co-glycolic acid) (PLGA), poly lactic acid (PLA), polyurethane, poly glycolic acid (PGA) or a combination thereof.

54. The formulation of claim 47, wherein the one or more hydrophobic polymers is polycaprolactone or polyvinyl acetate.

55. The formulation of claim 54, wherein the polycaprolactone polymer is present from about 5 to about 30% (w/w).

56. The formulation of claim 54, wherein the polyvinyl polymer is present from about 0% to about 20% (w/w)

57. The formulation of claim 47, wherein the ophthalmic drug is cyclosporine.

58. The formulation of claim 57, wherein the cyclosporine is present from about 60 to about 80% (w/w).

59. The formulation of claim 57, wherein the cyclosporine is present from about 65 to about 80% (w/w).

60. The formulation of claim 47, wherein the solid matrix composition comprises about 60 to about 240 μg of cyclosporine.

61. The formulation of claim 47, wherein the solid matrix composition is configured, when placed within the lacrimal canaliculus, to elute about 1 μg to about 3 μg of cyclosporine a day from about 2 weeks to about 8 weeks.

62. The formulation of claim 47, further comprising a sheath body disposed at least partially over the solid matrix.

63. The formulation of claim 47, wherein the ophthalmic drug is cyclosporine and the solid matrix comprises polycaprolactone and polyvinyl acetate.

64. The formulation of claim 47, wherein a first hydrophobic polymer is polycaprolactone and is present from 5 to 30% (w/w), a second hydrophobic polymer is polyvinyl acetate and is present from 0 to 20% (w/w), and the ophthalmic drug is cyclosporine and is present from 70 to 80% (w/w).

65. A sustained release ophthalmic formulation for topical delivery of an ophthalmic drug, comprising:
cyclosporine admixed with one or more hydrophobic polymers to form a solid matrix composition, wherein the composition is in the form of a drug core and configured for placement within a lacrimal canaliculus.

66. The formulation of claim 65, adapted to release the cyclosporine at therapeutically effective levels each day for a period of about two weeks to about 8 weeks.

67. The formulation of claim 65, wherein the drug core does not comprise silicone.

68. The formulation of claim 65, wherein the solid matrix does not comprise a nonionic surfactant selected from tyloxapol, a sorbitan ester, polyoxyethylene ethers, a polysorbate or a combination thereof.

69. The formulation of claim 65, wherein the drug core does not comprise PEG polymers.

70. The formulation of claim 65, wherein the drug core does not comprise a hydrophilic polymer selected from polyethylene glycol (PEG) polymers, acrylate-derivatized PEG (PEGDA) polymers, polysaccharide polymers, hydrophilic polyanhydrides or a combination thereof.

71. The formulation of claim 65, wherein the drug core does not comprise methacrylate polymers or monomers.

72. The formulation of claim 65, wherein the one or more hydrophobic polymers comprise silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate) (PVAc), poly(vinyl alcohol) (PVA), poly(vinyl ether), poly(vinyl ketone), poly(vinylpyrrolidone (PVP), poly(vinylpyridine), co-polymers thereof, or combinations thereof.

73. The formulation of claim 65, wherein the one or more hydrophobic polymers is selected from polyester, polyvinyl acetate, polycaprolactone, poly(D,L-lactic-co-glycolic acid) (PLGA), poly lactic acid (PLA), polyurethane, poly glycolic acid (PGA) or a combination thereof.

74. The formulation of claim 65, wherein the hydrophobic polymer is polycaprolactone or polyvinyl acetate.

75. The formulation of claim 74, wherein the polycaprolactone polymer is present from about 5 to about 30% (w/w).

76. The formulation of claim 74, wherein the polyvinyl acetate polymer is present from about 0 to about 20% (w/w).

77. The formulation of claim 65, wherein the cyclosporine is present from about 60 to about 80% (w/w).

78. The formulation of claim 65, wherein the cyclosporine is present from about 70 to about 80% (w/w).

79. The formulation of claim 65, wherein the solid matrix composition comprises about 60 to about 240 μg of cyclosporine.

80. The formulation of claim 65, wherein the drug core composition is configured, when placed within the lacrimal canaliculus, to elute about 1 μg to about 3 μg of the cyclosporine a day from about 2 weeks to about 8 weeks.

81. The formulation of claim 65, further comprising a sheath body disposed at least partially over the drug core.

82. The formulation of claim 65, wherein the drug core comprises polycaprolactone and polyvinyl acetate polymers.

83. The formulation of claim 65, wherein a first hydrophobic polymer is polycaprolactone and is present from 5 to 30% (w/w), a second hydrophobic polymer is polyvinyl acetate and is present from 0 to 20% (w/w), and the cyclosporine is present from 70 to 80% (w/w).

84. A lacrimal implant comprising:
a punctal plug comprising a plug body and a drug insert, wherein the insert comprises;
a drug core comprising the formulation according to any one of claim 47-83; and,
an impermeable sheath body partially covering the drug core, wherein the sheath body is configured to provide an exposed proximal end of the drug core in direct contact with tear fluid that releases an ophthalmic drug to the eye when the drug insert is disposed within a channel of the punctal plug and the punctal plug is inserted into the lacrimal canaliculus of a patient.

85. A method for delivering an ophthalmic drug to the eye for treatment of dry eye, comprising:
placing a lacrimal implant through a punctum and into a canalicular lumen of a patient, the implant comprising; a sustained release ophthalmic formulation according to any one of claim 47-83, wherein the ophthalmic drug is cyclosporine.

86. A solid matrix sustained release ophthalmic formulation for topical delivery of an ophthalmic drug, comprising:
a) one or more hydrophobic polymers; b) a nonionic surfactant and, c) the ophthalmic drug, wherein the formulation does not comprise a hydrophilic polymer and the formulation is adapted to release the ophthalmic drug at therapeutically effective levels each day for a period of about two weeks to about 8 weeks.

87. The formulation of claim 86, wherein the solid matrix does not comprise silicone.

88. The formulation of claim 86, wherein the nonionic surfactant is selected from tyloxapol, a sorbitan ester, polyoxyethylene ethers, a polysorbate or a combination thereof.

89. The formulation of claim 86, wherein the solid matrix does not comprise PEG polymers.

90. The formulation of claim 86, wherein the solid matrix does not comprise a hydrophilic polymer selected from polyethylene glycol (PEG) polymers, acrylate-derivatized PEG (PEGDA) polymers, polysaccharide polymers, hydrophilic polyanhydrides or a combination thereof.

91. The formulation of claim 86, wherein the solid matrix does not comprise methacrylate polymers or monomers.

92. The formulation of claim 86, wherein the one or more hydrophobic polymers comprise silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate) (PVAc), poly(vinyl alcohol) (PVA), poly(vinyl ether), poly(vinyl ketone), poly(vinylpyrrolidone (PVP), poly(vinylpyridine), co-polymers thereof, or combinations thereof.

93. The formulation of claim 86, wherein the one or more hydrophobic polymers is selected from polyester, poly(vinyl acetate) (PVAc), polycaprolactone, poly(D,L-lactic-co-glycolic acid) (PLGA), poly lactic acid (PLA), polyurethane, poly glycolic acid (PGA) or a combination thereof.

94. The formulation of claim 86, wherein the one or more hydrophobic polymers is polycaprolactone or polyvinyl acetate.

95. The formulation of claim 94, wherein the polycaprolactone polymer is present from about 5 to about 30% (w/w).

96. The formulation of claim 94, wherein the polyvinyl polymer is present from about 0% to about 20% (w/w)

97. The formulation of claim 86, wherein the ophthalmic drug is cyclosporine.

98. The formulation of claim 97, wherein the cyclosporine is present from about 60 to about 80% (w/w).

99. The formulation of claim 97, wherein the cyclosporine is present from about 65 to about 80% (w/w).

100. The formulation of claim 86, wherein the solid matrix composition comprises about 60 to about 240 μg of cyclosporine.

101. The formulation of claim 88, wherein the nonionic surfactant is polysorbate 80.

102. The formulation of claim 88, wherein the polysorbate 80 present in the drug core from about 0 to about 25% (w/w).

103. The formulation of claim 88, wherein the polysorbate 80 present in the drug core from about 3 to about 5% (w/w).

104. The formulation of claim 86, wherein the solid matrix composition is configured, when placed within the lacrimal canaliculus, to elute about 1 μg to about 3 μg of cyclosporine a day from about 2 weeks to about 8 weeks.

105. The formulation of claim 86, further comprising a sheath body disposed at least partially over the solid matrix.

106. The formulation of claim 86, wherein the ophthalmic drug is cyclosporine and the solid matrix comprises polycaprolactone and polyvinyl acetate.

107. The formulation of claim 86, wherein a first hydrophobic polymer is polycaprolactone and is present from 5 to 30% (w/w), a second hydrophobic polymer is polyvinyl acetate and is present from 0 to 20% (w/w), the nonionic surfactant is polysorbate 80 and is present from 3 to 5% (w/w), and the ophthalmic drug is cyclosporine and is present from 70 to 80% (w/w).

108. A sustained release ophthalmic formulation for topical delivery of an ophthalmic drug, comprising:
cyclosporine admixed with two or more hydrophobic polymers and a non-ionic surfactant to form a solid matrix composition, wherein the composition is in the form of a drug core and configured for placement within a lacrimal canaliculus.

109. The formulation of claim 108, adapted to release the cyclosporine at therapeutically effective levels each day for a period of about two weeks to about 8 weeks.

110. The formulation of claim 108, wherein the drug core does not comprise silicone.

111. The formulation of claim 108, wherein the nonionic surfactant is selected from tyloxapol, a sorbitan ester, polyoxyethylene ethers, a polysorbate or a combination thereof.

112. The formulation of claim 108, wherein the drug core does not comprise PEG polymers.

113. The formulation of claim 108, wherein the drug core does not comprise a hydrophilic polymer selected from polyethylene glycol (PEG) polymers, acrylate-derivatized PEG (PEGDA) polymers, polysaccharide polymers, hydrophilic polyanhydrides or a combination thereof.

114. The formulation of claim 108, wherein the drug core does not comprise methacrylate polymers or monomers.

115. The formulation of claim 108, wherein the two or more hydrophobic polymers comprise silicone, polycaprolactone (PCL), polyurethane, polyester, styrene, acrylate, methacrylate, acrylonitrile, maleic anhydride, polyamide, polyimide, polydiene, poly(ethylene terephthalate) (PET), polyethylene, polypropylene, polyether, poly(fluorocarbon) polymers, poly(vinyl acetal), poly(vinyl chloride), poly(vinyl acetate) (PVAc), poly(vinyl alcohol) (PVA), poly(vinyl ether), poly(vinyl ketone), poly(vinylpyrrolidone (PVP), poly(vinylpyridine), co-polymers thereof, or combinations thereof.

116. The formulation of claim 108, wherein the two or more hydrophobic polymers are selected from polyester, polyvinyl acetate, polycaprolactone, poly(D,L-lactic-co-glycolic acid) (PLGA), poly lactic acid (PLA), polyurethane, poly glycolic acid (PGA) or a combination thereof.

117. The formulation of claim 108, wherein the two hydrophobic polymers are polycaprolactone and polyvinyl acetate.

118. The formulation of claim 117, wherein the polycaprolactone polymer is present from about 5 to about 30% (w/w).

119. The formulation of claim 117, wherein the polyvinyl acetate polymer is present from about 0 to about 20% (w/w).

120. The formulation of claim 108, wherein the cyclosporine is present from about 60 to about 80% (w/w).

121. The formulation of claim 108, wherein the cyclosporine is present from about 70 to about 80% (w/w).

122. The formulation of claim 108, wherein the solid matrix composition comprises about 60 to about 240 μg of cyclosporine.

123. The formulation of claim 111, wherein the nonionic surfactant is polysorbate 80.

124. The formulation of claim 111, wherein the polysorbate 80 present in the drug core from about 0 to about 25% (w/w).

125. The formulation of claim 11, wherein the polysorbate 80 present in the drug core from about 3 to about 5% (w/w).

126. The formulation of claim 108, wherein the drug core composition is configured, when placed within the lacrimal canaliculus, to elute about 1 μg to about 3 μg of the cyclosporine a day from about 2 weeks to about 8 weeks.

127. The formulation of claim 108, further comprising a sheath body disposed at least partially over the drug core.

128. The formulation of claim 108, wherein the drug core comprises polycaprolactone and polyvinyl acetate polymers.

129. The formulation of claim 108, wherein a first hydrophobic polymer is polycaprolactone and is present from 5 to 30% (w/w), a second hydrophobic polymer is polyvinyl acetate and is present from 0 to 20% (w/w), the nonionic surfactant is polysorbate 80 and is present from 3 to 5%(w/w), and the cyclosporine is present from 70 to 80% (w/w).

130. A lacrimal implant comprising:
a punctal plug comprising a plug body and a drug insert, wherein the insert comprises;
a drug core comprising the formulation according to any one of claim 86-129; and,
an impermeable sheath body partially covering the drug core, wherein the sheath body is configured to provide an exposed proximal end of the drug core in direct contact with tear fluid that releases an ophthalmic drug to the eye when the drug insert is disposed within a channel of the punctal plug and the punctal plug is inserted into the lacrimal canaliculus of a patient.

131. A method for delivering an ophthalmic drug to the eye for treatment of dry eye, comprising:
placing a lacrimal implant through a punctum and into a canalicular lumen of a patient, the implant comprising; a sustained release ophthalmic formulation according to any one of claim 86-129, wherein the ophthalmic drug is cyclosporine.
